(19) United States
(12) Patent Application Publication
(10) Pub. No.: US 2006/0281776 A1
(43) Pub. Date: Dec. 14, 2006

(54) Isotope labeled camptothecin derivatives

(75) Inventors: Danilo Giribone, Milano (IT); Romualdo Forino, Milano (IT); Natale Barbugian, Milano (IT); Erminia Fontana, Nerviano (IT)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/540,081

(22) PCT Filed: Dec. 19, 2003
(86) PCT No.: PCT/EP03/14801

§ 371 Date: Jul. 19, 2006

(30) Foreign Application Priority Data
Dec. 20, 2002 (EP) 02080413.4

(51) Int. Cl. A61K 31/4745; C07D 471/14

(52) U.S. Cl. 514/283; 546/52

(57) ABSTRACT

The present invention provides isotope stable labeled camaptothecin analogs including irinotecan and SN-38, a process for their preparation and their use as internal standard in analytical methods.

### FIELD OF THE INVENTION

The present invention pertains to the field of isotopically labeled compounds useful in the absorption, distribution, metabolism, excretion (ADME), pharmacokinetic and pharmacodynamic studies. The invention relates to, in particular, the preparation of stable labeled camptothecin analogs starting from commercially available stable labeled precursors at high isotopic enrichment.

### BACKGROUND OF THE INVENTION

Camptothecin is an alkaloid derived from the Chinese tree. Camptotheca acuminata. Camptothecin and its derivatives are unique in their ability to inhibit DNA Topoisomerase, by stabilizing a covalent reaction intermediate termed the cleavable complex which ultimately causes tumor cell death. Topoisomerase is responsible for the winding/unwinding of the supercoiled DNA composing the chromosomes. If the chromosomes cannot be unwound, transcription of DNA message cannot occur and the protein cannot be synthesized, it ultimately causes cell death. Application of camptothecin in clinic is limited due to serious side effects and poor water-solubility. At present, some camptothecin analogs, either semi-synthetic or synthetic drug based on camptothecin, have been applied cancerous therapy such as topotecan and irinotecan.

Since its approval in the United States in 1996, irinotecan hydrochloride trihydrate (CPT-11, CAMPTOSAR®, injection, Pharmacia Corp.; Peapack, N.J.) has undergone extensive clinical evaluation. In the past five years, the focus of development has evolved from evaluation of single-agent activity in refractory disease settings to evaluation of front-line irinotecan-based combination chemotherapy regimens and integration of irinotecan into combined modality regimens. Important studies have been performed clarifying the role of irinotecan treating colorectal and other gastrointestinal cancers, small cell and non-small cell lung cancer, and a variety of other malignancies.

CPT-11 has shown activity against a variety of tumour types, particularly refractory colorectal tumours, and it is used for the treatment of various forms of cancer. Its primary use is in the treatment of colon cancer, particularly advanced colon cancer. It is also of interest for treatment of other cancers, however, and mention is made of cancers of the lung, the stomach and the pancreas.

The antitumor activity of CPT-11 is attributed to an active metabolite, 7-ethyl-10-hydroxy 20(S) camptothecin (SN-38), which is produced after enzymatic cleavage by carboxylesterases in the liver, small intestine and plasma.

Accurate, sensitive and specific measurement of campthotecin analogs and their metabolites such as, for example, irinotecan and its metabolite SN-38 may allow to carry out precise pharmacokinetic and pharmacodynamic analysis of these products in biological samples such as, for example, animal and human plasma, urine, bile, tissues and in vitro cell culture media.

For example, one of the most convenient method for daily routine analysis was obtained by using automated sample handling procedure followed by liquid chromatography (LC) and mass spectrometry detection (MS). One crucial aspect of a reliable and validated analytical method is the availability of a suitable internal standard. The addition of known amount of an internal standard to the unknown sample is a well-known and widely used procedure that can compensate for losses of the compound of interest during sample workup. According to this approach, any loss of the compound of interest can be determined by the loss of an equivalent fraction of internal standard. The precision and accuracy of this approach is strongly dependent on the structural similarity between the compound of interest and the internal standard. As a consequence it is generally agreed that the stable isotopically labeled analogues with the same molecular structure of a compound are the best internal standards for liquid chromatography-mass spectrometry (LC-MS) assay. In addition the internal standard should have preferably a molecular weight at least three mass units higher than that of the non-labeled compound of interest.

There is therefore a need of stable isotope labeled camptobthecin analogs especially irinotecan and its active metabolite SN-38, in order to improve the accuracy, sensitivity and specificity of the analytical methods to determine the non labeled parent drugs or their metabolites in biological samples. The following invention fulfills such a need by providing stable labeled camptothecin derivatives comprising irinotecan and SN-38.

### DETAILED DESCRIPTION OF THE INVENTION

It is therefore an object of the present invention stable labeled camptothecin analogs and a method for their preparation starting from commercially available stable labeled precursors at high isotopic enrichment and non-labeled intermediates that can be synthesized according to well known methods.

In particular, the present invention provides stable labeled camptothecin analogs of formula (I) 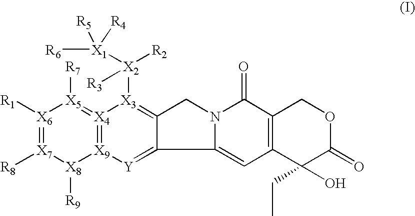
wherein

each of R₂, R₃, R₄, R₅, R₆, R₇, R₈ and R₉ independently represents ²H or H;

each of X₁, X₂, X₃, X₄, X₅, X₆, X7, X₈ and X₉ independently represents ¹³C or C;

Y is ¹⁵N or N; and

R₁ is hydroxyl group or a group of formula (i) 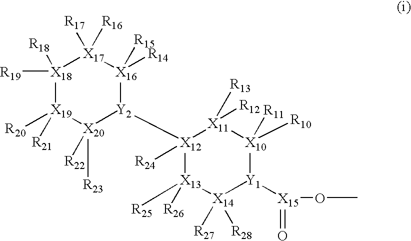
wherein

each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,

each of X₁₀, X₁₁, X₁₂, X₁₃, X₁₄, X₁₅, X₁₆, X₁₇, X₁₈, X₁₉ and X₂₀ independently represents ¹³C or C,

each of Y₁ and Y₂ independently represents ¹⁵N or N;

with the proviso that at least one of R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, Y, Y₁ and Y₂ is isotopically labeled;

or a pharmaceutically acceptable salt thereof.

Pharmaceutically acceptable salts of a compound of formula (I) are, for example, salts with an inorganic or organic acid. In general, inorganic acids and organic acids are physiologically acceptable and are selected, for example, from hydrochloric acid, hydrobromic acid, hydriodic acid, sulfuric acid, phosphoric acid, nitric acid, acetic acid, propionic acid, 1-ascorbic acid, tartaric acid, citric acid, lactic acid, maleic acid, fumaric acid, methanesulfonic acid, toluenesulfonic acid and benzenesulfonic acid.

Preferred pharmaceutically acceptable salt of a compound of formula (I) is hydrochloride salt.

In a first preferred embodiment, the compounds of formula (I) are compounds of formula (I) as defined above wherein, subject to the above proviso, R₁ is a hydroxyl group.

In a second preferred embodiment, the compounds of formula (I) are compounds of formula (I) wherein, subject to the above proviso, R₁ is a group of formula (i) as defined above.

In a third preferred embodiment, the compounds of formula (I) are compounds of formula (I) wherein, subject to the above proviso, R₂, R₃, R₄, R₅, R₆, R₇, R₈ and R₉ are all H, X₁, X₂, X_{3,} X₄, X₅, X₆, X₇, X₈ and X₉ are all C, Y is N and R₁ is a group (i) as defined above.

In a forth preferred embodiment, the compounds of formula (I) are compounds of formula (I) wherein, subject to the above proviso, each of R₂, R₃, R₄, R₅, R₆, R₇, R₈ and R₉ independently represents ²H of H, each of X₁, X₂, X₃, X₄, X₅, X₆, X₇, X₈ and X₉ independently represents ¹³C or C, Y is ¹⁵N or N, R₁ is a hydroxyl group or a group of formula (i) wherein R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ are all H, X₁₀, X₁₁, X₁₂, X₁₃, X₁₄, X₁₅, X₁₆, X₁₇, X₁₈, X₁₉ and X₂₀ are all C and Y₁ and Y₂ are N.

The preferred compounds according to the present invention are the compounds of formula (I) having the structures (I′) and (I″)

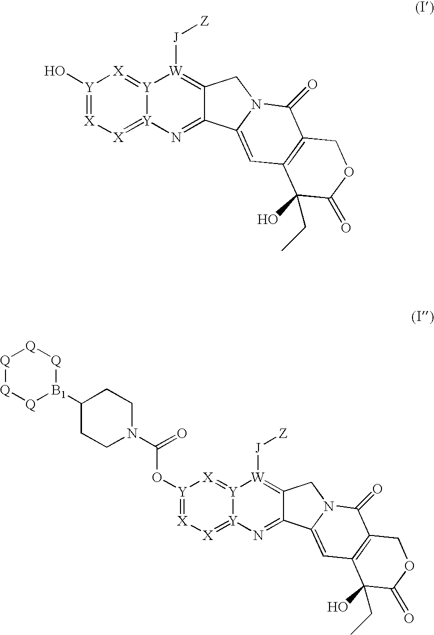

wherein X, Y, W, J, Z, Q and B₁ are as defined in the following Table 1 and Table 2 regarding the structures (I′) for the compounds 1 to 13 and the structures (I″) for the compounds 14 to 54 respectively and, if the case, their pharmaceutically acceptable salts.

TABLE 1

| compound | Z | J | W | X | Y |
|---|---|---|---|---|---|
| 1 | CD₃ | CH₂ | C | CH | C |
| 2 | CD₃ | CH₂ | C | ¹³CH | ¹³C |
| 3 | CD₃ | CD₂ | C | CH | C |
| 4 | CD₃ | CD₂ | C | ¹³CH | ¹³C |
| 5 | ¹³CH₃ | ¹³CH₂ | ¹³C | ¹³CH | ¹³C |
| 6 | CH₃ | CH₂ | ¹³C | ¹³CH | ¹³C |
| 7 | CH₃ | CH₂ | C | ¹³CH | ¹³C |
| 8 | CH₃ | ¹³CH₂ | C | ¹³CH | ¹³C |
| 9 | ¹³CH₃ | CH₂ | C | ¹³CH | ¹³C |
| 10 | CH₃ | CH₂ | ¹³C | ¹³CH | ¹³C |
| 11 | CH₃ | CH₂ | ¹³C | CH | C |
| 12 | CH₃ | ¹³CH₂ | C | CH | C |
| 13 | CD₃ | CH₂ | C | CH | C |

TABLE 2

| compound | Z | J | W | X | Y | Q | B₁ |
|---|---|---|---|---|---|---|---|
| 14 | CH₃ | CH₂ | C | CH | C | CD₂ | N |
| 15 | CH₃ | CH₂ | C | CH | C | CH₂ | ¹⁵N |
| 16 | CD₃ | CH₂ | C | CH | C | CD₂ | N |
| 17 | CD₃ | CH₂ | C | CH | C | CH₂ | ¹⁵N |
| 18 | CD₃ | CH₂ | C | CH | C | CH₂ | N |
| 19 | CD₃ | CH₂ | C | ¹³CH | ¹³C | CD₂ | N |
| 20 | CD₃ | CH₂ | C | ¹³CH | ¹³C | CH₂ | ¹⁵N |
| 21 | CD₃ | CH₂ | C | ¹³CH | ¹³C | CH₂ | N |
| 22 | CD₃ | CD₂ | C | CH | C | CD₂ | N |
| 23 | CD₃ | CD₂ | C | CH | C | CH₂ | ¹⁵N |
| 24 | CD₃ | CD₂ | C | CH | C | CH₂ | N |
| 25 | CD₃ | CD₂ | C | ¹³CH | ¹³C | CD₂ | N |
| 26 | CD₃ | CD₂ | C | ¹³CH | ¹³C | CH₂ | ¹⁵N |
| 27 | CD₃ | CD₂ | C | ¹³CH | ¹³C | CH₂ | N |
| 28 | ¹³CH₃ | ¹³CH₂ | ¹³C | ¹³CH | ¹³C | CD₂ | N |
| 29 | ¹³CH₃ | ¹³CH₂ | ¹³C | ¹³CH | ¹³C | CH₂ | ¹⁵N |
| 30 | ¹³CH₃ | ¹³CH₂ | ¹³C | ¹³CH | ¹³C | CH₂ | N |
| 31 | CH₃ | CH₂ | ¹³C | ¹³CH | ¹³C | CD₂ | N |
| 32 | CH₃ | CH₂ | ¹³C | ¹³CH | ¹³C | CH₂ | ¹⁵N |
| 33 | CH₃ | CH₂ | ¹³C | ¹³CH | ¹³C | CH₂ | N |
| 34 | CH₃ | CH₂ | C | ¹³CH | ¹³C | CD₂ | N |
| 35 | CH₃ | ¹³CH₂ | C | ¹³CH | ¹³C | CH₂ | ¹⁵N |
| 36 | CH₃ | ¹³CH₂ | C | ¹³CH | ¹³C | CH₂ | N |
| 37 | CH₃ | ¹³CH₂ | C | ¹³CH | ¹³C | CD₂ | N |
| 38 | CH₃ | ¹³CH₂ | C | ¹³CH | ¹³C | CH₂ | ¹⁵N |
| 39 | CH₃ | ¹³CH₂ | C | ¹³CH | ¹³C | CH₂ | N |
| 40 | ¹³CH₃ | CH₂ | C | ¹³CH | ¹³C | CD₂ | N |
| 41 | ¹³CH₃ | CH₂ | C | ¹³CH | ¹³C | CH₂ | ¹⁵N |
| 42 | ¹³CH₃ | CH₂ | C | ¹³CH | ¹³C | CH₂ | N |
| 43 | CH₃ | CH₂ | ¹³C | ¹³CH | ¹³C | CD₂ | N |
| 44 | CH₃ | CH₂ | ¹³C | ¹³CH | ¹³C | CH₂ | ¹⁵N |
| 45 | CH₃ | CH₂ | ¹³C | ¹³CH | ¹³C | CH₂ | N |
| 46 | CH₃ | CH₂ | ¹³C | CH | C | CD₂ | N |
| 47 | CH₃ | CH₂ | ¹³C | CH | C | CH₂ | ¹⁵N |
| 48 | CH₃ | CH₂ | ¹³C | CH | C | CH₂ | N |
| 49 | CH₃ | ¹³CH₂ | C | CH | C | CD₂ | N |
| 50 | CH₃ | ¹³CH₂ | C | CH | C | CH₂ | ¹⁵N |
| 51 | CH₃ | ¹³CH₂ | C | CH | C | CH₂ | N |
| 52 | CD₃ | CH₂ | C | CH | C | CD₂ | N |
| 53 | CD₃ | CH₂ | C | CH | C | CH₂ | ¹⁵N |
| 54 | CD₃ | CH₂ | C | CH | C | CH₂ | N |

In the present specification the capital letter “D” means deuterium (²H).

The present invention also provides a process for the preparation of a stable labeled camptothecin analog of formula (I) wherein

R₁ is a hydroxyl group,

each of R₂, R₃, R₄, R₅, R₆, R₇, R₈ and R₉ independently represents ²H or H,

each of X₁, X₂, X₃, X₄, X₅, X₆, X₇, X₈ and X₉ independently represents ¹³C or C, and

Y is ¹⁵N or N,

with the proviso that at least one of R₂, R₃, R₄, R₅, R₆, R₇, R₈, R₉, X₁, X₂, X₃, X₄, X₅, X₆, X₇, X₈, X₉ and Y is isotopically labeled,

which comprises:

(a) reacting a compound of formula (II) 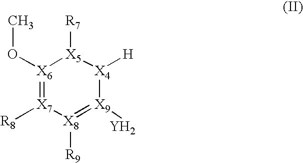
wherein

each of R₇₁ R₈ and R₉ independently represents ²H or H.

each of X₄, X₅, X₆, X₇, X₈ and X₉ independently represents ¹³C or C, and

Y is ¹⁵N or N,

with a compound of formula (III) 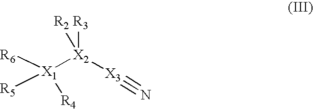
wherein

each of R₂, R₃, R₄, R₅ and R₆ independently represents ²H or H, and

each of X₁, X₂ and X₃ independently represents ¹³C or C, to obtain the compound of formula (IV) 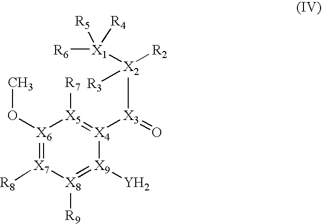
wherein

each of R₂, R₃, R₄, R₅, R₆, R₇, R₈, R₉, X₁, X₂, X₃, X₄, X₅, X₆, X₇, X₈, X₉ and Y, are as above described,

so that at least one of R₂, R₃, R₄, R₅, R₆, R₇, R₈, R₉, X₁, X₂, X₃, X_{4,} X₅, X₆, X₇, X₈, X₉ and Y is isotopically labeled;

(b) cleaving a compound of formula (IV) to obtain a compound of formula (V) 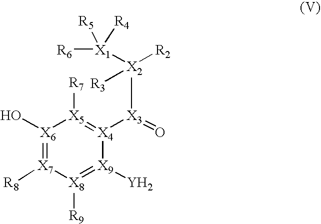
wherein

R₂, R₃, R₄, R₅, R₆, R₇, R₈, R₉, X₁, X₂, X₃, X₄, X₅, X₆, X₇, X₈, X₉ and Y are as above described for the compound (IV); and

(c) reacting a compound of formula (V) with the compound of formula (VI)

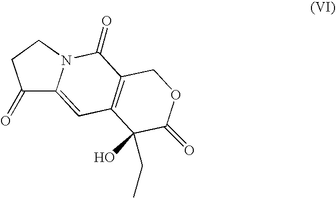

to obtain the desired compound of formula (I).

In a further aspect, the present invention provides a process for preparing a compound of formula (I) wherein each of R₂, R₃, R₄, R₅, R₆, R₇, R₈ and R₉ independently represents ²H or H,

each of X₁, X₂, X₃, X₄, X₅, X₆, X₇, X₈ and X₉ independently represents ¹³C or C,

Y is ¹⁵N or N, and

R₁ is a group of formula (i) 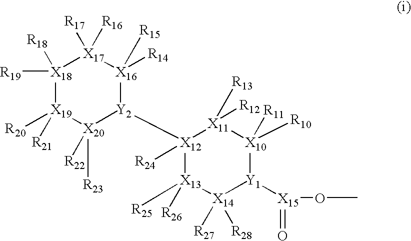
wherein

each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,

each of X₁₀, X₁₁, X₁₂, X₁₃, X₁₄, X₁₅, X₁₆, X₁₇, X₁₈, X₁₉ and X₂₀ independently represents ¹³C or C, and

each of Y₁ and Y₂ independently represents ¹⁵N or N,

with the proviso that at least one of R₂, R₃, R₄, R₅, R₆, R₇, R₈, R₉, X₁, X₂, X₃, X₄, X₅, X₆, X₇, X₈, X₉ and Y is isotopically labeled, and that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀, Y₁ and Y₂ is isotopically labeled,

which comprises:

(d) reacting a compound of formula (I) as obtained in step (c) above with a compound of formula (VII) 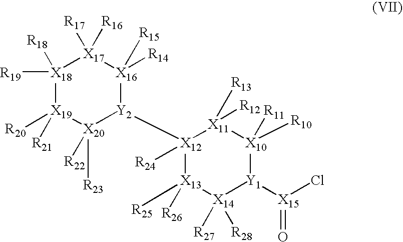
wherein

each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H.

each of X₁₀, X₁₁, X₁₂, X₁₃, X₁₄, X₁₅, X₁₆, X₁₇, X₁₈ and X₂₀ independently represents 13 C or C, and

each of Y₁ and Y₂ independently represents ¹⁵N or N.

with the proviso that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀, Y₁ and Y₂ is isotopically labeled, to obtain the desired compound of formula (I).

In a still further aspect, the present invention provides a process for preparing a compound of formula (I) wherein R₂, R₃, R₄, R₅, R₆, R₇, R₈ and R₉ are all H; X₁, X₂, X₃, X₄, X₅, X₆, X₇, X₈and X₉ are all C, Y is N and R₁ is a group of formula (i) 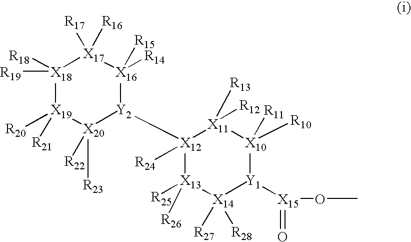
wherein

each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,

each of X¹⁰, X¹¹, X₁₂, X₁₃, X₁₄, X₁₅, X₁₆, X₁₇, X₁₈, X₁₉ and X₂₀ independently represents ¹³C or C, and

each of Y₁ and Y₂ independently represents ¹⁵N or N,

with the proviso that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀, Y₁ and Y₂ is isotopically labeled,

which comprises:

(e) reacting the compound of formula 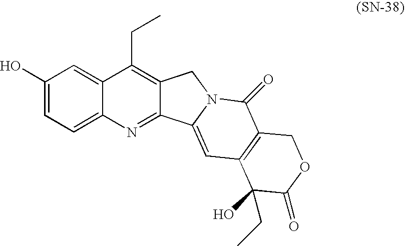
with a compound of formula (VII) as above described to obtain the desired compound of formula (I), and optionally converting it into a pharmaceutically acceptable salt thereof.

In a still another aspect, the present invention provides a process for preparing a compound of formula (I) wherein each of R₂, R₃, R₄, R₅, R₆, R₇, R₈, R₉, X₁, X₂, X₃, X₄, X₅, X₆, X₇, X₈, X₉and Y, are as above described, with the proviso that at least one of R₂, R₃, R₄, R₅, R₆, R₇, R₈, R₉, X₁, X₂, X₃, X₄, X₅, X₆, X₇, X₈, X₉ and Y is isotopically labeled, and R₁ is a group of formula (i) wherein R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ are all H and X₁₀, X₁₁, X₁₂, X₁₃, X₁₄, X₁₅, X₁₆, X₁₇, X₁₈, X₁₉ and X₂₀ are all C, Y₁and Y₂ are N,

which comprises:

(f) reacting a compound of formula (I) as obtained in step (c) above with the compound of formula

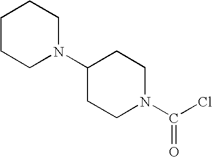

to obtain the desired compound of formula (I), and optionally converting it into a pharmaceutically acceptable salt thereof.

The intermediate compound of formula (VII) can be prepared with a process, which comprises:

(g) reacting a compound of formula (VIII) 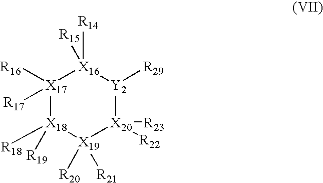
wherein

each of R₁₄, R₁₅, R₁₆, R₁₇, R₁₈, R₁₉, R₂₀, R₂₁, R₂₂, R₂₃ and R₂₉ represents independently ²H or H, and

each of X₁₆, X₁₇, X₁₈, X₁₉ and X₂₀ represents independently ¹³C or C, and Y₂ is ¹⁵N or N,

with a compound of formula (IX) 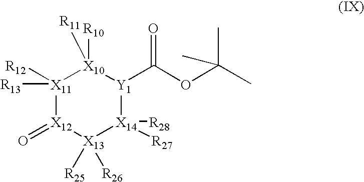
wherein

each of R₁₀, R₁₁, R₁₂, R₁₃, R₁₄, R₂₅, R₂₆, R₂₇ and R₂₈ represents independently ²H or H, and

each of X₁₀, X₁₁, X₁₂, X₁₃ and X₁₄ represents independently ¹³C or C, and

Y₁ is ¹⁵N or N,

to obtain a compound of formula (X) 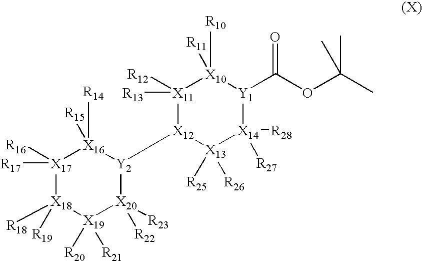
wherein

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄X₁₆, X₁₇, X₁₈, X₁₉, X₂₀, Y₁, and Y₂ are as above described for the compounds (VIII) and (IX),

so that at least one of R₁₀, R₁₁, R₁₂, R₁₃, R₁₄, R₁₅, R₁₆, R₁₇, R₁₈, R₁₉, R₂₀, R₂₁, R₂₂, R₂₃, X₁₀, X₁₁, X₁₂, X₁₃, X₁₄, X₁₆, X₁₇, X₁₈, X₁₉, X₂₀, Y₁ and Y₂ is isotopically labeled;

(h) cleaving a compound of formula (X) to obtain a compound of formula (XI) 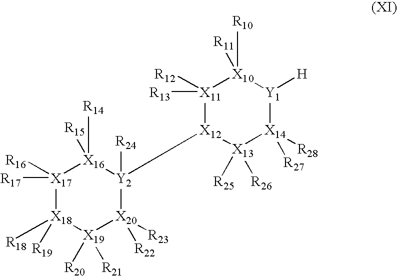
wherein R₁₀, R₁₁, R₁₂, R₁₃, R₁₄, R₁₅, R₁₆, R₁₇, R₁₈, R₁₉, R₂₀, R₂₁, R₂₂, R₂₃, X₁₀, X₁₁, X₁₂, X₁₃, X₁₄, X₁₆, X₁₇, X₁₈, X₁₉, X₂₀, Y₁, and Y₂ are as above described for the compound (X);

(i) reacting a compound of formula (XI) with a suitable haloacylating agent of formula (XIII) 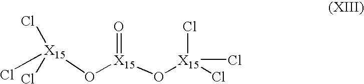
wherein X₁₅ is ¹³C or C,

to obtain a compound as a hydrochloride salt of formula (VII) 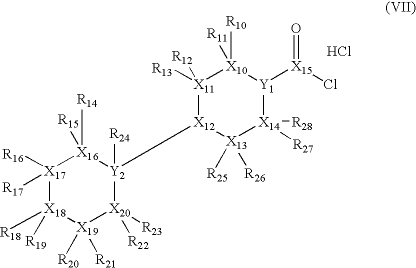
wherein

R₁₀, R₁₁, R₁₂, R₁₃, R₁₄, R₁₅, R₁₆, R₁₇, R₁₈, R₁₉, R₂₀, R₂₁, R₂₂, R₂₃, X₁₀, X₁₁, X₁₂, X₁₃, X₁₄, X₁₆, X₁₇, X₁₈, X₁₉, X₂₀, Y₁ and Y₂ are as above described for the compound (X) and, converting a compound of formula (VII) into the corresponding free base with the same formula (VII)

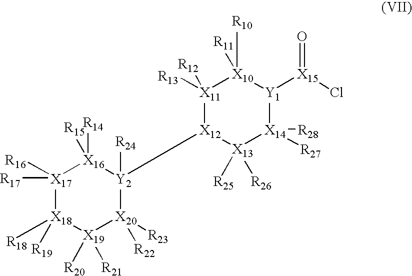

The processes described above are particularly advantageous as they enable the selective preparation of a variety of compounds of formula (I) isotopically labeled. In addition, they enable the preparation of the desired derivatives in high yields and with a high degree of isotopic enrichment.

According to step (a) of the above process, the reaction between a compound of the formula (II) with a compound of the formula (III) is carried out under an inert atmosphere, for example under nitrogen, by heating preferably at 90° C. a mixture of the above two compounds with BCl₃ and a Lewis acid, such as for example AlCl₃ in an inert organic solvent, such as for example a mixture of dichloromethane, toluene and dichloroethane. It is preferred to pre-mix a compound of formula (II) and BCl₃ in a preferred molar ratio of 1 to 1.1 at low temperature, such as for example 4° C. It is preferred to pre-mix a compound of formula (III) and the Lewis acid in a molar ratio of 1 to 2÷20, preferably 1 to 3, at low temperature, such as for example below 10° C. The molar ratios between a compound of formula (II) and of a compound of a formula (III) are preferably 1 to 4. The progress of the reaction is checked by an analytical method, for example thin layer chromatography or high performance liquid chromatography or mass spectrometry, and the disappearance of the compound of formula (II) is complete generally within about 4 hours. The reaction mixture is then added with water and heated, -for example at 80° C., in order to allow the formation of a compound of formula (IV). The progress of this stage of the reaction is checked by an analytical method, for example thin layer chromatography or high performance liquid chromatography or mass spectrometry, and is complete generally within about 30 minutes. The two-phase mixture is cooled, preferably at room temperature, for example at 25° C. The organic phase is extracted with an acidic aqueous solution, for example 1N hydrochloric acid. All the collected aqueous phases are pooled and added with a base, for example a 35% aqueous solution of NaOH, up to basic pH, for example 8.9. This mixture is extracted with a non-water miscible solvent, such as for example dichloromethane, and all the organic extracts are pooled. This solution is dried over an inorganic salt, such as for example sodium sulfate, and the solvent is evaporated, for example with a rotating evaporator. The so obtained crude material containing a compound of formula (IV) is preferably purified by using techniques well known in the art. For example, preparative-column chromatography using silica gel along with appropriate eluants such as mixtures of organic solvents may be used to effectively purify the desired compound so as the following cleavage of a compound of formula (IV) is successfully carried out.

According to step (b) of the above process, a compound of formula (IV) is treated with an agent capable of cleaving the alkyl-aryl ether bond, such as for example a solution of bromidric acid, at high temperature, such as for example at 110° C. The concentration of the bromidric acid is preferably 48%. The concentration of a compound of formula (IV) into the bromidric acid solution is preferably 0.4M. The progress of this reaction is checked by an analytical method, for example thin layer chromatography or high performance liquid chromatography or mass spectrometry, and His complete generally within about 5 hours. The above mixture is cooled, preferably at 5° C., and the precipitated compound of formula (V) is filtered and washed with cold water, for example at 5° C. The above wet material is suspended in water and the mixture is added with a base, such as for example 32% aqueous NaOH, in order to obtain a pH value around 10. The mixture is cooled, preferably at 5° C., and the precipitated compound of formula (V) is filtered, for example with a glass filtering funnel, washed with cold water, for example at 5° C., and dried, for example under vacuum.

According to step (c) of the above process, the reaction between a compound of formula (V) and the compound of formula (VI) is carried out by heating, for example at about 100° C., the above compounds in an inert organic solvent, for example toluene, in the presence of acidic substances, for example organic acids such as, for example, p-toluensulphonic acid and acetic acid. During the above reaction the forming water is continuously removed, for example with a stream of nitrogen. The molar ratio between a compound of formula (V) and a compound of formula (VI) is preferably 1 to 1. The catalytic amount of the p-toluenesulphonic acid is preferably 6 mg/mmol of compound of formula (V). The progress of this reaction is checked by an analytical method, for example thin layer chromatography or high performance liquid chromatography or mass spectrometry, and is complete generally within about 7 hours. The above mixture is then cooled, preferably at 25° C., and allowed to stay under stirring for several hours, for example 18 hours, obtaining the precipitation of a compound of formula (I) that is then filtered and dried, for example under vacuum. The so obtained crude material containing a compound of formula (I) is preferably purified by using techniques well known in the art. For example, by slurry in an organic solvent that is capable to dissolve the impurities rather than the desired product of the formula (I), such as for example absolute ethanol, followed by filtration, for example with a sintered glass filtering funnel, and drying for example under vacuum.

According to step (d) of the above process, the reaction between a compound of formula (I) as obtained in step (c) and a compound of formula (VII) as free base as obtained in step (i) is carried out at room temperature, for example at 25° C., in the presence of a base and a solvent. The base can be also the solvent such as for example in the case of using pyridine. The molar ratio between a compound of the formula (I) and a compound of formula (VII) is 1 to 1÷2, preferably 1 to 1.5. The progress of this reaction is checked by an analytical method, for example thin layer chromatography or high performance liquid chromatography or mass spectrometry, and is complete generally within about 1 hour. The volatile products are removed with well known methods, for example by means of distillation. The obtained compound of formula (I) as a hydrochloride salt is precipitated by adding an inert organic solvent in which it is not soluble, such as for example n-hexane, and then filtrated, for example with a sintered glass filtering funnel, and dried for example under vacuum. The so obtained crude material containing a compound of formula (I) is preferably purified by using techniques well known in the art, for example, by precipitating the pure corresponding free base by adjusting the pH of the hydrochloride salt aqueous solution to a value of 7 by adding a basic inorganic compound such as, for example, di-potassium hydrogen phosphate. After filtration, for example with a sintered glass filtering funnel, and drying for example under vacuum, a pure compound of formula (I) as a free base is obtained. The hydrochloride salt of a compound of formula (I) can be obtained by dissolving the free base into a hydrochloric acid solution, for example 1N aqueous HCl, and evaporating the solvent for example under vacuum, preferably by lyophilization. The molar ratio between a compound of formula (I) as free base and the acid is preferably 1 to 1.3.

According to step (e) of the above process, the reaction between SN-38 and a compound of formula (VII) as free base as obtained in step (i) is carried out in the same way as stated for the above described step (d) obtaining a compound of formula (I).

According to step (f) of the above process, the reaction between a compound of formula (I) as obtained in step (c) and the commercially available compound of formula 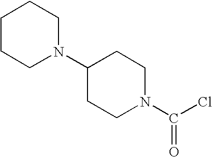
is carried out in the same way as stated for the above described step (d) obtaining the compounds of formula (I).

According to step (g) of the above process, the reaction between a compound of formula (VIII) and a compound of formula (IX) is carried out under an inert atmosphere, for example under nitrogen, by means of a reducing agent, such as for example NaBH₃CN in the presence of a Lewis acid catalyst, such as for example Titanium(IV)isopropoxyde, in an organic solvent, such as for example ethanol, at room temperature, such as for example at 25° C. It is preferably to pre-mix a compound of formula (VIII) and (IX) with the Lewis acid catalyst without solvents and before adding the reducing agent. The molar ratio among a compound of the formula (VIII), (IX), and the Lewis acid catalyst is preferably 1 to 1 to 1.25. The equivalent ratio between a compound of the formula (VIII) and the reducing agent preferably 1 to 2. The progress of this reaction is checked by an analytical method, for example thin layer chromatography or high performance liquid chromatography or mass spectrometry, and is complete generally within about 20 hours. At the end of the reaction the mixture is added with water, stirred for several hours, for example 4 hours, at room temperature, for example at 25° C., and then filtered, for example through a sintered-glass filtering funnel recovering the organic solution containing a compound of the formula (X). The crude material containing a compound of formula (X) is recovered by removing the solvents, for example under reduced pressure. The so obtained crude material is preferably purified by using techniques well known in the art. For example, preparative-column chromatography using silica gel along with appropriate eluants such as mixtures of organic solvents may be used to effectively purify the desired compound so as the following cleavage of a compound of formula (X) is successfully carried out.

The compounds of formula (VIII) and (IX) are commercially available compounds or can be obtained by applying well-known procedures in the art.

According to step (h) of the above process, a labeled compound of formula (X) are cleaved by means of an acidic agent, such as for example trifluoroacetic acid, in an inert organic solvent, such as for example dichloroethane. The reaction is carried out under an inert atmosphere, for example under nitrogen, at room temperature, for example 25° C. The concentration of the acidic reagent into the reaction mixture is about 30÷70, preferably 45% by volume. The molar ratio of a compound of formula (X) and the acidic reagent is about 1 to 4÷10, preferably 1 to 6. 5. The progress of this reaction is checked by an analytical method, for example thin layer chromatography or high performance liquid chromatography or mass spectrometry, and is complete generally within about 2 hours. At the end of the reaction the mixture is preferably diluted with a base solution in a solvent which is not miscible with the reaction solvent, such as for example 32% aqueous NaOH, up to basic condition of the aqueous layer, for example pH 12÷13. The aqueous layer is extracted with a non-water miscible solvent capable of dissolving the compounds of the formula (XI) such as for example an organic solvent such as for example dichloromethane. The solution containing a compound of formula (XI) is preferably dried, for example with an inorganic salt such as for example sodium sulphate, and filtered, for example through a sintered glass filtering funnel. The crude material containing a compound of formula (XI) is recovered after solvent evaporation to dryness, for example under reduced pressure.

According to step (i) of the above process, a labeled compound of the formula (XI) is converted into the corresponding carbamoyl chloride hydrochloride of formula (VII) by means of a haloacylating agent, such as for example triphosgene of formula (XIII). The reaction is carried out at low temperature, for example below 10° C. and preferably at about 4° C., in an inert organic solvent, such as for example toluene, under an inert atmosphere, for example under nitrogen. The equivalent ratio between a compound of formula (XI) and the haloacylating agent is about 1 to l to 5 preferably 1 to 1.8. The progress of this reaction is checked by an analytical method, for example thin layer chromatography or high performance liquid chromatography or mass spectrometry, and is complete generally within about 30 minutes. At the end of the reaction the mixture is filtered, for example through a sintered glass filtering funnel, under an inert atmosphere, for example under nitrogen obtaining the crude material containing a compound of formula (VII) which is recovered as a solid after solvent evaporation to dryness, for example under reduced pressure. The crude material containing a compound of formula (VII) is preferably purified, before the subsequent step (e) or (f), by using techniques well known in the art. For example, by dissolving a compound of formula (VII) in a solvent that does not dissolve the impurities present into the crude material, such as for example dichloromethane. The suspension is filtered, for example through a sintered glass filtering funnel, under an inert atmosphere, for example under nitrogen, and the collected solution is concentrated, for example at reduced pressure under an inert atmosphere, for example under nitrogen. The concentrated solution containing a compound of formula (VII) is dripped under an inert atmosphere, for example under nitrogen, to a solvent that poorly dissolves a compound of formula (VII), such as for example methylcyclopentane. At the end of the precipitation the mixture is filtered, for example through a sintered glass filtering funnel, under an inert atmosphere, for example under nitrogen obtaining the material containing a compound of formula (VII) as a hydrochloride salt which is recovered as a solid after solvent evaporation to dryness, for ,example under reduced pressure. A compound of formula (VII) as a hydrochloride salt can be converted into the corresponding free base of formula (VII) by treating its solution into an inert organic solvent that is capable to dissolve the free base, such as for example dichloromethane, with a base dissolved in water, for example an aqueous solution of an inorganic base, preferably 1M potassium carbonate, at low temperature, for example 0° C. The free base of the formula (VII) is recovered from the organic layer by evaporating the solvent, for example at reduced pressure.

SN-38 is commercially available or can be obtained by procedures well known in the art, for example following the procedure of K. E. Henegar et al. J. Org. Chem. 62(1997) 6588-97.

The optional salification of a compound of formula (I) may be carried out by conventional methods.

A further object of the present invention is the use of a stable labeled camptothecin analog of formula (I) for ADME studies.

Another object of the present invention is the use of a stable labeled camptothecin analog of formula (I) as an internal standard in an analytical method for the quantitative detection of the corresponding unlabeled camptothecin derivative in a biological sample.

A biological sample is preferably a biological fluid, e.g., animal and human plasma, urine, bile, tissues and in vitro cell culture media.

In a particular aspect, the present invention provides the use of a stable labeled camptothecin analog having the above-identified structures (I′) and (I″) as defined in TABLE 1 and TABLE 2 above or a pharmaceutically acceptable salt thereof, as an internal standard in an analytical method for the quantitative detection of the corresponding unlabeled camptothecin derivative in a biological sample.

The following Examples 1-17 illustrate the preparation of the compounds (1) to (13) of TABLE 1 of the present invention following the synthetic SCHEME 1 reported below. 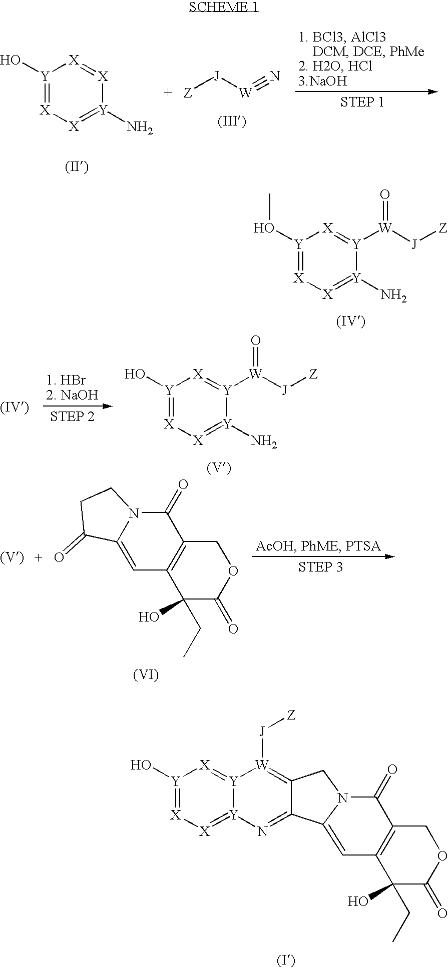
AcOH=acetic acid, DCE=dichloroethane, DCM=dichloromethane, PhMe=toluene, PTSA=p-toluenesulfonic acid. The meanings of the sustituents X, Y, W, J and Z are defined in the Examples 1-17.

### EXAMPLE 1

### Crude Labeled 1-(2-amino-5-methoxy-phenyl)-propan-1-one, Compound of Formula (IV′) Where X═CH, Y═W═C, J=CH₂, Z=CD₃.

To a cold (4° C.) stirred solution of boron trichloride in dry dichloromethane (0.92M, 15 ml) prepared under nitrogen, a solution of the compound of formula (II′) where X═CH, Y═C (1.5719 g) in toluene (15 ml) was slowly added. This 15 mixture, called reactive A, was kept at 4° C. under nitrogen with stirring before its use.

To a cold (10 ° C.) stirred solution of the labeled compound of formula (III′) where, W═C, J=CH₂, Z=CD₃ (3.1 g) in dichloroethane (10 ml) prepared under nitrogen, aluminum trichloride (2.0720 g) was slowly added. This mixture was slowly heated to 75° C. and kept under these conditions while the whole amount of reactive A was rapidly added. A gentle stream of nitrogen was allowed to pass through the reactor and the external temperature was increased up to 110° C. When the distillation of dichloromethane and acidic vapors ceased the reaction temperature became stable at 90° C. After about 4 hours the end of the reaction was checked by (i) TLC on silica gel 60 with fluorescent indicator at 254 nm plates with thickness of 0.25 mm eluted with dichloromethane-ethyl acetate mixture 98:2 by volume, developing agents=UV light at 254, 336 nm and aqueous permanganate solution; and by (ii) HPLC on C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection wavelength=225 nm) and the heating was discontinued. The reaction mixture was cooled to about 10° C. and water (30 ml) was added over 10 minutes under stirring. The clear two-phase brown mixture was heated at 85° C. for 30 minutes, and then cooled to room temperature. After phase separation the dark brown organic phase was extracted with further 1N HCl (25 ml×5 times) then discarded. All the yellow aqueous acidic phases were pooled and slowly added with 35% NaOH up to pH 11. The clear basic aqueous solution was extracted with dichloromethane until -a colorless organic extraction phase was obtained (15 ml×9 times). The organic extracts were pooled, dried over Na₂SO₄ and the solvent evaporated under vacuum obtaining an orange yellow oily residue containing the compound of formula (IV′) where X═CH, Y═W═C, J=CH₂, Z=CD₃. The purity of about 65% was assessed by HPLC (C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection wavelength=225 nm), the retention time (Rt=5.50 minutes) was the same as the retention time of an authentic non-labeled sample.

### EXAMPLE 2

### Purification of the Crude Material Containing Labeled 1-(2-amino-5-methoxy-phenyl)-propan-1-one, Compound of Formula (IV′) Where X═CH, Y═W═C, J=CH₂, Z=CD₃.

The crude material containing the compound of formula (IV′) where X═CH, Y═W═C, J=CH₂, Z=CD₃, prepared as described in EXAMPLE 1, was diluted with dichloromethane (15 ml) and flash-chromatographed on a SiO₂ column (130×6.5 ID mm) eluting with a mixture of dichloromethane-ethylacetate (980:20 by vol., total elution volume about 2.2 1). Fractions of about 100 ml were collected and checked by (i) TLC on silica gel 60 with fluorescent indicator at 254 nm plates with thickness of 0.25 mm eluted with dichloromethane-ethyl acetate mixture 98:2 by volume, developing agents=UV light at 254, 336 nm and aqueous permanganate solution) and by (ii) HPLC on C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection wavelength =225 nm). All the fractions containing the pure compound of interest (from 3 to 7) were combined and the solvent evaporated under vacuum to dryness. The compound of formula (IV′) where X═CH, Y═W═C, J=CH₂, Z=CD₃ (0.9816 g) was obtained as a bright yellow solid, >90% chemically pure. The purity was assessed by HPLC (C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection wavelength=225 nm), the retention time (Rt=5.50 minutes) was the same as the retention time of an authentic non-labeled sample. The mass spectrum was recorded using the electrospray ionization technique (ESI) with positive ion detection. The ESI mass spectrum showed the protonated molecular ions at m/z 183 amu.

### EXAMPLE 3

### Labeled 1-(2-Amino-5-hydroxy-phenyl)-propan-1-one, Compound of Formula (V′) Where X═CH, Y═W═C, J=CH₂, Z=CD₃.

The compound of formula (IV′ where X═CH, Y═W═C, j=CH₂, Z=CD₃ (0.9816 g), prepared as described in EXAMPLE 1 and purified for example as described in EXAMPLE 2, was suspended in a cold (4° C.) solution of 48% bromidric acid (15 ml) under nitrogen. After refluxing for about 5 hours the end of the reaction was checked (by HPLC on C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection wavelength 225 nm). The reaction mixture was cooled to about 5° C., stirred for 1 hour and filtered obtaining a light brown solid which was washed with the mother liquor and cold (4° C.) water (2×0.75 ml). The wet cake was suspended in water (4.7 ml), slowly added with 32% NaOH up to neutrality and then with 1N NaOH up to pH≈10. The suspension was cooled (4° C.) and stirred for 30 minutes then was filtered and the solid was washed with the mother liquor and cold (4° C.) water (2×1.5 ml). The solid product was dried under vacuum at room temperature for 18 hours obtaining the compound of formula (V′) where X═CH, Y═W═C, J=CH₂, Z=CD₃ as beige solid (0.8 g). The purity higher than 99% was assessed by HPLC (C-8 reverse phase column along with eluants as mixtures of water-acetonitrile-trifluoroacetic acid from 90:10:0.1 to 10:90:0.1 by volume, linear gradient over 13 minutes and 8 minutes of isocratic elution, detection. wavelength=225 nm), the retention time (Rt=2.30 minutes) was the same as the retention time of an authentic non-labeled sample. The mass spectrum of the title compound was recorded using the electrospray ionization technique (ESI) with positive ion detection. The ESI mass spectrum showed the protonated molecular ions at m/z 169. The NMR spectrum recorded in CDCl₃at 400 MHz showed the following signals expressed as chemical shifts (ppm) : 8.60-8.62, d; 7.08-7.10, m; 6.76-6.82, m; 6.58-6.60, m; 2.82-2.86, d.

### EXAMPLE 4

### Compound of Formula (I′) Where X═CH, Y═W═C, J=CH₂, Z=CD₃ crude labeled SN-38 (1)

A mixture of the compound of formula (V′ where X═CH, Y═W═C, J=CH₂, Z=CD₃ (0.560 g), prepared as described in EXAMPLE 3, the compound of formula (VI) (0.868 g), p-Toluensulphonic acid monohydrate (20 mg), glacial acetic acid (3.5 ml) and toluene (14.0 ml) was stirred at 101° C. under a gentle stream of nitrogen to remove the water formed. After 7 hrs the end of the reaction was checked (by HPLC on C-18 reverse phase column along with eluants as a mixture of water-acetonitrile-trifluoroacetic acid at a constant ratio of 70:30:0.2 by volume). The mixture was diluted with toluene (9.4 ml) then cooled to room temperature and stirred overnight to complete the crystallization. The precipitate was filtered, washed with toluene and dried under vacuum at 45° C. obtaining a solid containing the compound of formula (I′) where X═CH, Y═W═C, J=CH₂, Z=CD₃. The purity of about 90% was assessed by HPLC (C-18 reverse phase column along with eluants as mixture of water-acetonitrile-trifluoroacetic acid 70:30:0.2 by volume, 30 minutes of isocratic elution, detection wavelength=260 nm), the retention time (Rt=6.7 minutes) was the same as the retention time of an authentic, non-labeled sample. The mass spectrum of the title compound was recorded using the electrospray ionization technique (ESI) with positive ion detection. The ESI mass spectrum showed the protonated molecular ions at m/z 396 amu.

### EXAMPLE 5

### Purification of the Crude Material Containing Labeled SN-38 (1)

The dry crude material containing (1), prepared as described in EXAMPLE 4, was suspended in absolute ethanol and stirred thoroughly. After 1 hour the suspension was filtered and the solid was dried under vacuum at room temperature for 18 hours obtaining the compound (1) as whitish powder (1.13 g). The purity grater than 99% was assessed by HPLC (C-18 reverse phase column along with eluants as mixture of water-acetonitrile-trifluoroacetic acid 70:30:0.2 by volume, 30 minutes of isocratic elution, detection wavelength=260 nm), the retention time (Rt=6.7 minutes) was the same as the retention time of an authentic non-labeled sample. The mass spectrum of the title compound was recorded using the electrospray ionization technique (ESI) with positive ion detection. The ESI mass spectrum showed the protonated molecular ions ([M+H]⁺) at m/z 396 amu. The NMR spectrum recorded in DMSO-d5 at 500 MHz showed the following signals expressed as chemical shifts (ppm): 10.29, s; 8.02, d; 7.41, m; 7.25, s; 6.47, b; 5.42, s; 5.29,s; 3.07, s; 1.85, m; 0.88, t.

### EXAMPLE 6

### Compound of Formula (I′) Where Z=CD₃, J=CH₂, W═C, X═¹³CH, Y═¹³C, Labeled SN-38 (2)

Starting from the labeled compound of formula (III′) where Z=CD₃, J=CH₂, W═C and the labeled compound of the formula (II′) where X═¹³CH, Y═¹³C and following the procedure described in EXAMPLES 1 to 5, the labeled compound of formula (I′) where Z=CD₃, J=CH₂, W═C, X═¹³CH, Y═¹³C (2) can be obtained.

### EXAMPLE 7

### Compound of Formula (I′) Where Z=CD₃, J=CD₂, W═C, X═CH, Y═C, Labeled SN-38 (3)

Starting from the labeled compound of formula (III′) where Z=CD₃, J=CD₂, W═C and the compound of the formula (II′) where X═CH, Y═C and following the procedure described in EXAMPLES 1 to 5, the labeled compound of formula (I′) where Z=CD₃, J=CD₂ W═C, X═CH, Y═C (3) can be obtained.

### EXAMPLE 8

### Compound of Formula (I′) Where Z=CD₃, J=CD₂, W═C, X═¹³CH, Y═¹³C; Labeled SN-38 (4)

Starting from the labeled compound of formula (III′) where Z=CD₃, J=CD₂, W═C and the labeled compound of the formula (II′) where X═¹³CH, Y═¹³C and following the procedure described in EXAMPLES 1 to 5, the labeled compound of formula where Z=CD₃, J=CD₂, W═C, X═¹³CH(4), Y═¹³C can be obtained.

### EXAMPLE 9

### Compound of formula (I′) Where Z=¹³CH₃, J=¹³CH₂, W═¹³C, X═¹³CH, Y═¹³C, labeled SN-38 (5)

Starting from the labeled compound of formula (III′) where Z=¹³CH₃, J=¹³CH₂, W═¹³C and the compound of the formula (II′) where X═¹³CH, Y═¹³C and following the procedure described in EXAMPLES 1 to 5, the labeled compound of formula where Z=¹³CH₃, J=¹³CH₂, W═¹³C, X═¹³CH, Y═¹³C (5) can be obtained.

### EXAMPLE 10

### Compound of Formula (I′) Where Z=¹³CH₃, J=¹³CH₂, W═¹³C, X═CH, Y═C, Labeled SN-38 (6)

Starting from the labeled compound of formula (III′) where Z=¹³CH₃, J=¹³CH₂, W═¹³C and the compound of the formula (II′) where X═CH, Y═C and following the procedure described in EXAMPLES 1 to 5, the labeled compound of formula (I′) where Z=¹³CH₃, J=¹³CH₂, W═¹³C, X═CH, Y═C (6) can be obtained.

### EXAMPLE 11

### Compound of Formula (I′) Where Z=CH₃, J=CH₂, W═C, X═¹³CH, Y═¹³C Labeled SN-38 (7)

Starting from the compound of formula (III′) where Z=CH₃, J=CH₂, W═C and the labeled compound of the formula (II′) where X═¹³CH, Y═¹³C and following the procedure described in EXAMPLES 1 to 5, the labeled compound of formula (I′) where Z=CH₃, J=CH₂, W═C, X═¹³CH, Y═¹³C (7) can be obtained.

### EXAMPLE 12

### Compound of Formula (I′) Where Z=CH₃, J=¹³CH₂, W═C, X═¹³CH, Y═¹³C Labeled SN-38 (8)

Starting from the labeled compound of formula (III′) where Z=CH₃, J=³CH₂, W═C and the labeled compound of the formula (II′) where X═¹³CH, Y═¹³C and following the procedure described in EXAMPLES 1 to 5, the labeled compound of formula (I′) where Z=CH₃, J=¹³CH₂, W═C, X═¹³CH, Y=¹³C (8) can be obtained.

### EXAMPLE 13

### Compound of Formula (I′) Where Z=¹³CH₃, J=CH₂, W═C, X═¹³CH, Y═¹³C Labeled SN-38 (9)

Starting from the labeled compound of formula (III′) where Z=¹³CH₃, J=CH₂, W═C and the labeled compound of the formula (II′) where X═¹³CH, Y═¹³C and following the procedure described in EXAMPLES 1 to 5, the labeled compound of formula where Z=¹³CH₃, J=CH₂, W=C, X═¹³CH, Y═¹³C (9) can be obtained.

### EXAMPLE 14

### Compound of Formula (I′) Where Z=CH₃, J=CH₂, W═¹³C, X═¹³CH, Y═¹³C Labeled SN-38 (10)

Starting from the labeled compound of formula (III′) where Z=CH₃, J=CH₂, W═¹³C and the labeled compound of the formula (II′) where X═¹³CH, Y═¹³C and following the procedure described in EXAMPLES 1 to 5, the labeled compound of formula where Z=CH₃, J=CH₂, W═¹³C, X═¹³CH, Y═¹³C (10) can be obtained.

### EXAMPLE 15

### Compound of Formula (I′) Where Z=CH₃, J=CH₂, W═¹³C, X═CH, Y═C Labeled SN-38 (11)

Starting from the labeled compound of formula (III′) where Z=CH₃, J=CH₂, W═¹³C and the compound of the formula (II′) where X═CH, Y═C and following the procedure described in EXAMPLES 1 to 5, the labeled compound of the formula where Z=CH₃, J=CH₂, W═¹³C, X═CH, Y═C (11) can be obtained.

### EXAMPLE 16

### Compound of Formula (I′) Where Z=CH₃, J=¹³CH₂, W═C, X═CH, Y═C Labeled SN-38 (12)

Starting from the labeled compound of formula (III′) where Z=CH₃, J=¹³CH₂, W═C and the compound of the formula (II′) where X═CH, Y═C and following the procedure described in EXAMPLES 1 to 5, the labeled compound of formula where Z=CH₃, J=¹³CH₂, W═C, X═CH, Y═C (12) can be obtained.

### EXAMPLE 17

### Compound of Formula (I′) Where Z=¹³CH₃, J=CH₂, W═C, X═CH, Y═C Labeled SN-38 (13)

Starting from the labeled compound of formula (III′) where Z=¹³CH₃, J=CH₂, W═C and the compound of the formula (II′) where X═CH, Y═C and following the procedure described in EXAMPLES 1 to 5, the labeled compound of formula where Z=¹³CH₃, J=CH₂, W═C, X═CH, Y═C (13) can be obtained.

The following Examples 18-70 illustrate the preparation of the compounds (14) to (54) of TABLE 2 of the present invention following the synthetic SCHEME 2 reported below. 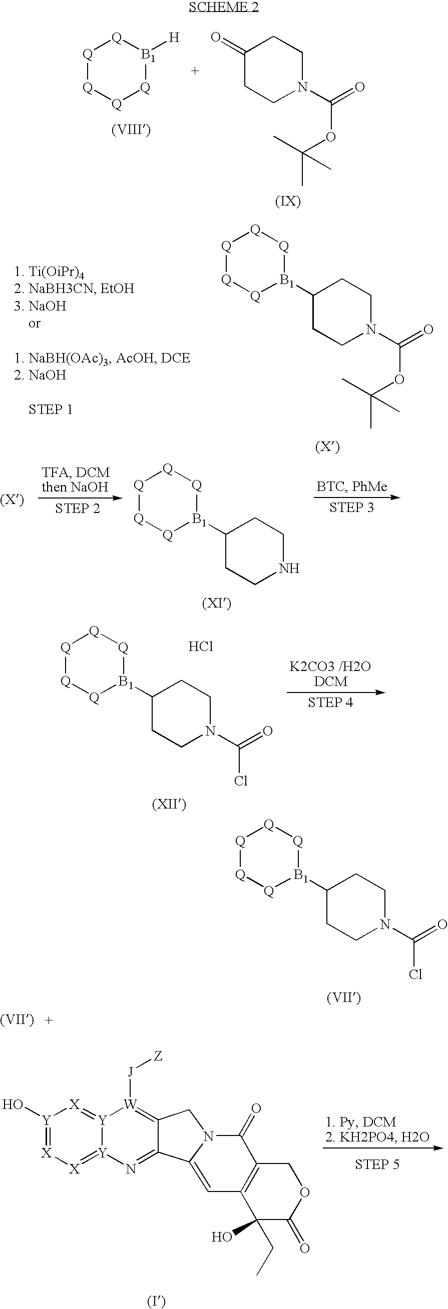 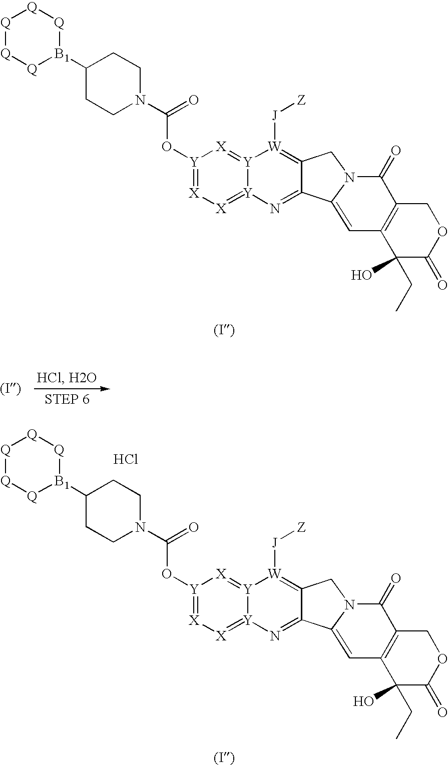
EtOH=ethanol, ACOH=acetic acid, DCE=dichloroethane, DCM=dichloromethane, TFA=trifluoroacetic acid, BTC=bis-trichloromethyl carbonate (triphosgene), PhMe=toluene, Py=pyridine. The meanings of the substituents X, Y, W, J, Z, Q and B₁ are as defined in the Examples 18-70.

### EXAMPLE 18

### Crude Labeled [1,4′]Bipiperidinyl-1′-carboxylic acid tert-butyl ester, Compound of Formula (X′) Where Q=CD₂, B₁═N.

The labeled compound of formula (VIII′) where Q=CD₂, B₁═N (2 ml), the compound of formula (IX) (4.01 g) and Ti(OiPr)₄ (7.44 ml) were stirred under nitrogen at room temperature for 1 hour. The mixture was diluted with absolute ethanol (10 ml) then NaBH₃CN (0.085 g) was added along with further absolute ethanol (10 ml) and stirred at room temperature under nitrogen. After 19 hours the suspension was diluted with water (4 ml) and stirred at room temperature. After 4.5 hours the mixture was filtered and the white precipitate was washed with ethanol (4×15 ml) collecting all the filtrates. The ethanol phases were pooled, evaporated under reduced pressure and the residue was dissolved in dichloromethane (30 ml). The organic solution was washed with 1 N NaOH (3×30 ml), dried over sodium sulfate and evaporated to dryness under reduced pressure obtaining an orange oily residue (5.1 g) which contained the compound of formula (X′) where Q=CD₂, B₁═N (checked by TLC on silica gel 60 with fluorescent indicator at 254 nm plates with thickness of 0.25 mm eluted with methanol-ethyl acetate mixture 98:2 by volume, developing agents=UV light at 254, 336 nm and aqueous permanganate solution). The mass spectrum of the above material was recorded using the electrospray ionization technique (ESI) with positive ion detection. The ESI mass spectrum showed the protonated molecular ions at m/z 279 amu.

### EXAMPLE 19

### Crude Labeled [1,4′]Bipiperidinyl-1′carboxylic acid tert-butyl ester, Compound of Formula (X′) where Q=CD₂, B₁═N.

To a solution of the compound of formula (IX) (3.99 g) in dichloroethane (60 ml) was added the compound of formula (VIII′) where Q=CD₂, B₁═N (2 ml) under nitrogen with stirring at room temperature. After 10 minutes NaB(OAc)₃ (6.1792 g) and glacial acetic acid (1.15 ml) were added to the mixture obtaining a suspension that was stirred under nitrogen at room temperature. After 48 hours the mixture was diluted with dichloromethane (30 ml) and added with 1N NaOH (60 ml). After 10 minutes of stirring the organic layer was separated, washed with 1N NaOH (3×30 ml) and dried over sodium sulfate. The solution volume was reduced to 30 ml under reduced pressure then washed with further 1N NaOH (3×50 ml) and dried over sodium sulfate. After evaporation to dryness a yellow-orange oily residue containing the compound of formula (X′) where Q=CD₂, B₁═N was obtained (5.0 g (checked by TLC on silica gel 60 with fluorescent indicator at 254 nm plates with thickness of 0.25 mm eluted with methanol-ethyl acetate mixture 98:2 by volume, developing agents=UV light at 254, 336 nm and aqueous permanganate solution). The mass spectrum of the above material was recorded using the electrospray ionization technique (ESI) with positive ion detection. The ESI mass spectrum showed the protonated molecular ions at m/z 279 amu.

### EXAMPLE 20

### Purification of the Crude Material Containing Labeled [1,4′]Bipiperidinyl-1′-carboxylic acid tert-butyl ester, Compound of Formula (X′) Where Q=CD₂, B₁═N.

The crude material (5 g) containing the compound of formula (X′) where Q=CD₂, B₁═N, prepared as described in EXAMPLE 18 or EXAMPLE 19, was diluted with a mixture of ethylacetate-methanol (95:5 by volume, 20 ml) and flash-chromatographed on a SiO₂ pre-packed column (70×40 ID mm) eluting with a mixture of ethylacetate-methanol (95:5 by volume, total elution volume about 2.2 1). Fractions of about 50 ml were collected and checked (by TLC on silica gel 60 with fluorescent indicator at 254 nm plates with thickness of 0.25 mm eluted with methanol-ethyl acetate mixture 98:2 by volume, developing agents=UV light at 254, 336 nm and aqueous permanganate solution). All the fractions containing the compound of interest (from 3 to 39) were combined and the solvent evaporated under vacuum. The obtained yellow oily residue (4.40 g) was divided in two equal portions that were diluted with a mixture of ethylacetate-methanol (95:5 by volume, 7 ml). Both solutions containing the compound of formula (X′) where Q=CD₂, B₁═N, were flash-chromatographed on a SiO₂ pre-packed column (140×40 ID mm) eluting with a mixture of ethylacetate-methanol (95:5 by volume, total elution volume about 2.5 1). Fractions of about 50 ml were collected and checked (by TLC on silica gel 60 with fluorescent indicator at 254 nm plates with thickness of 0.25 mm eluted with methanol-ethyl acetate mixture 98:2 by volume, developing agents=UV light at 254, 336 nm and aqueous permanganate Resolution). All the fractions containing the pure compound of interest (from 12 to 45 for both columns) were combined and the solvent evaporated under vacuum obtaining the pure compound of formula (X′) where Q=CD₂, B₁═N, (2.73 g) as colorless oil. The mass spectrum of the above material was recorded using the electrospray ionization technique (ESI) with positive ion detection. The ESI mass spectrum showed the protonated molecular ions at m/z 279 amu.

### EXAMPLE 21

### Labeled [1,4′]Bipiperidinyl, Compound of Formula (XI′) Where Q=CD₂, B₁═N.

A cold (4° C.) solution of the compound of formula (X′) where Q=CD₂, B₁═N (2.73 g), prepared as described in EXAMPLE 18 or EXAMPLE 19 and eventually purified as described EXAMPLE 20, in dichloromethane (10 ml) was slowly added with trifluoroacetic acid (7.5 ml) under nitrogen with stirring. The reaction mixture was then stirred at 25° C. After 2 hours the end of the reaction was checked (by TLC on silica gel 60 with fluorescent indicator at 254 nm plates with thickness of 0.25 mm eluted with dichloromethane-ethyl acetate mixture 98:2 by volume, developing agents=UV light at 254, 336 nm and aqueous permanganate solution), the mixture was cooled to 4° C. and slowly added under vigorous stirring with 32% NaOH up to pH 12÷13 of the aqueous phase. The mixture was diluted with dichloromethane (20 ml) and water (50 ml) and the organic phase was separated and collected. The aqueous phase was further extracted with dichloromethane (4×30 ml). All the organic extracts were pooled, dried over Na₂SO₄ and filtered. After solvent evaporation to dryness under reduced pressure at room temperature for 18 hours the compound of formula (XI′) where Q=CD₂, B₁═N was recovered as a white solid (1.15 g). The mass spectrum of the above material was recorded using the electrospray ionization technique (ESI) with positive ion detection. The ESI mass spectrum showed the protonated molecular ions ([M+H]⁺) at m/z 179 amu. The NMR spectrum recorded in DMSO-d5 at 500 MHz showed the following signals expressed as chemical shifts (ppm): 3.28, b; 2.96-2.85, m; 2.30-2.39 m; 2.11-2.23, m; 1.52-1.64, m; 1.15-1.29, m.

### EXAMPLE 22

### Crude Labeled [1,4′]Bipiperidinyl-1′-carbonyl chloride hydrochloride, Compound of Formula (XII′) Where Q=CD₂, B₁═N.

To a cold (4° C.) solution of triphosgene (0.5878 g) in toluene (32.5 ml) a solution of the compound of formula (XI′) where Q=CD₂, B₁═N (0.6503 g), prepared as described in EXAMPLE 21, in dry toluene (3.7 ml) was slowly added under nitrogen with vigorous stirring. After 30 minutes of stirring at 4° C. under nitrogen the reaction mixture showed no presence starting material of formula (XI′) where Q=CD₂, B₁═N (checked by HPLC on amino phase column along with eluant as mixture of water, containing 3.4 g/l of KH₂PO₄, 5.0 g/l KCl and H₃PO₄ up to pH2.2, and acetonitrile in a constant ratio of 30:70 by volume). The suspension was filtered under nitrogen and the white precipitate was washed with toluene (2×2 ml) and hexane (2×5 ml) under nitrogen. After drying the under vacuum at room temperature for 3 hours the crude material (0.90 g) containing the compound of formula (XII′) where Q=CD₂, B₁═N was obtained as a white solid. The purity of about 70% was assessed by HPLC (on amino phase column along with eluant as mixture of water, containing 3.4 g/l of KH2PO4, 5.0 g/l KCl and H₃PO₄ up to pH=2.2, and acetonitrile in a constant ratio of 30:70 by volume), the retention time (Rt=4.3 minutes) was the same as the retention time of an authentic non-labeled sample. The mass spectrum of the title compound was recorded using the electrospray ionization technique (ESI) with positive ion detection. The ESI mass spectrum showed the protonated molecular ions ([M+H]⁺) at m/z 241 amu.

### EXAMPLE 23

### Purification of the Crude Material Containing Labeled [1,4′]Bipiperidinyl-1′-carbonyl chloride hydrochloride, Compound of Formula (XII′) Where Q=CD₂, B₁═N.

The crude material containing the compound of formula (XII′) where Q=CD₂, B₁═N (about 0.90 g), prepared as described in EXAMPLE 22, was added under nitrogen with dichloromethane (10 ml) and the resulting suspension was stirred at room temperature for 15 minutes. After adding a filter-aid agent (0.3 g) and stirring for further 10 minutes, the suspension was filtered under nitrogen collecting the filtrate. The solid was washed with dichloromethane (2×3 ml) collecting the washings. All the clear dichloromethane phases were pooled and concentrated under a stream of nitrogen at reduced pressure up to a total volume of about 4 ml. This solution was slowly dripped under nitrogen at room temperature into methylcyclohexane (25 ml) with vigorous stirring. The white suspension was stirred for 20 minutes at room temperature under nitrogen then was filtered under nitrogen. The solid was washed with methylcyclohexane (3×10 ml) then dried at room temperature under vacuum for 13 hours. The compound of formula (XII′) where Q=CD₂, B₁═N (about 0.67 g) was obtained as a white solid. The purity of about 90% was assessed by HPLC (on amino phase column along with eluant as mixture of water, containing 3.4 g/l of KH2PO4, 5.0 g/l KCl and H₃PO₄ up to pH2.2, and acetonitrile in a constant ratio of 30:70 by volume), the retention time (Rt=4.30 minutes) was the same as the retention time of an authentic non-labeled sample. The mass spectrum of the title compound was recorded using the electrospray ionization technique (ESI) with positive ion detection. The ESI mass spectrum showed the protonated molecular ions ([M+H]⁺) at m/z 241 amu and also other characteristic ions ([M-Cl]⁺) at m/z 205 amu. The NMR spectrum recorded in DMSO-d5 at 500 MHz showed the following signals expressed as chemical shifts (ppm): 9.55-9.74 (HCl), s; 4.10-4.34, m; 3.33-3.46 m; 2.93-3.22, m; 2.04-2.17, m; 1.59-1.75, m.

### EXAMPLE 24

### Labeled [1,4′]Bipiperidinyl-1′-carbonyl chloride, Compound of Formula (VII′) Where Q=CD₂, B₁═N.

To a cooled (0°÷−5° C.) dichloromethane solution of the compound of formula (XII′) where Q=CD₂, B₁═N (about 180 mg), prepared as described in EXAMPLE 22 and purified as described in EXAMPLE 23, an excess of a water solution of 1M potassium carbonate was added. The organic layer containing the compound of formula (VII′) where Q=CD₂, B₁═N was separated and was partially evaporated to 1.5 ml under reduced pressure. A concentrated solution of the compound of formula (VII′) where Q=CD₂, B₁═N was obtained.

### EXAMPLE 25

### Labeled [1,4′]Bipiperidinyl-1′-carbonyl chloride Compound of Formula (VII′) Where Q=CH₂, B₁═¹⁵NH.

Starting from the compound of formula (VIII′) where Q=CH₂, B₁═¹⁵N and following the procedure described in EXAMPLES 18 to 24, the labeled compound of the formula (VII′) where Q=CH₂, B₁═¹⁵NH can be obtained.

### EXAMPLE 26

### Compound of Formula (II′) where Q=CD₂, B₁═N, Y═C, X═CH, W═C, J=CH₂, Z=CH₃, Crude Labeled CPT-11 (14)

To a stirred mixture of the compound of formula (I′) where Y═C, X═CH, W═C, J=CH₂, Z=CH₃ (176 mg) in Pyridine (2.6 ml), a dichloromethane solution of the compound of formula (VII′) where Q=CD₂, B₁═N, (156 mg), prepared as described in EXAMPLE 24, was dropped over about 1 hr at room temperature. The reaction mixture was stirred for about 30 minutes at room temperature, and then evaporated under reduced pressure at 40° C. The residue was added with toluene (7.0 ml) and the mixture was distilled in order to remove the residual pyridine. To the residue, n-Hexane (10 ml) was added and the suspension was stirred until homogeneous slurry was obtained. The solid was isolated by filtration, washed with n-Hexane (15 ml) and dried obtaining the crude material containing the compound of formula (I″) where Q=CD₂, B₁═N, Y═C, X═CH, W═C, J=CH₂, Z=CH₃ as a hydrochloride salt as a brownish powder.

### EXAMPLE 27

### Precipitation of the Labeled CPT-11 (14) as a Free Base.

The crude material containing the compound of formula (14) as a hydrochloride salt, prepared as described in EXAMPLE 26, was dissolved in water (3.0.ml) and the value of the pH was adjusted to 7.0 by adding di-Potassium hydrogen phosphate. The precipitated free base of the compound was isolated by filtration and washed with water (10 ml).

### EXAMPLE 28

### Precipitation of the Labeled CPT-11 (14) as a Hydrochloride Salt.

To a solution of the crude free base prepared as described in EXAMPLE 27, in water (9.0 ml), 1N hydrochloric acid (0.47 ml, about 1.3 equivalents) was added. The acidic solution was filtered and the filtrate was evaporated under reduced pressure at 40° C. to a smaller volume (about 1.2 ml). The product was isolated from the aqueous solution by freeze-drying. The labeled CPT-11 (14) was obtained as a white solid (256 mg). The purity greater than 98.8% was assessed by HPLC (on C18 reverse phase column along with eluant as mixture of water-acetonitrile-trifluoroacetic acid in a constant ratio of 71:29:0.2 by volume), the retention time (Rt=7.60 minutes) was the same as the retention time of an authentic non-labeled sample. The mass spectrum of the title compound was recorded using the electrospray ionization technique (ESI) with positive ion detection. The ESI mass spectrum showed the protonated molecular ions at m/z 597 amu. The NMR spectrum recorded in DMSO-d5 at 500 MHz showed the following signals expressed as chemical shifts (ppm) : 9.89, b; 8.20, d; 8.00, d; 7.69, dd; 7.23, s; 6.52, b; 5.45, S; 5.35, s; 4.42, m; 4.21, m; 3.45, m; 3.20, q; 2.95-3.16, m; 2.17, m; 1.65-1.95, m; 1.30, t; 0.89, t.

### EXAMPLE 29

### Compound of Formula (I″) where Q=CD₂, B₁═N, Y═C, X═CH, W═C, J=CH₂, Z=CD₃ Crude Labeled CPT-11 (16)

To a stirred mixture of the labeled SN-38 (1) prepared as described in EXAMPLE 4 and purified as described in EXAMPLE 5, in Pyridine (7.4 ml), a dichloromethane solution of the compound of formula (VII′) where Q=CD₂, B₁═N (421 mg), prepared as described in EXAMPLE 24, was dropped over about 1 hr at room temperature. The reaction mixture was stirred for about 30 minutes at room temperature, and then evaporated under reduced pressure at 40° C. The residue was added with toluene (30.0 ml) and the mixture was distilled in order to remove the residual Pyridine. To the residue, n-Hexane (30 ml) was added and the suspension was stirred until homogeneous slurry was obtained. The solid was isolated by filtration, washed with n-Hexane (30 ml) and dried obtaining the crude material containing the compound of formula (I″) (16) where Q=CD₂, B₁═N, Y═C, X═CH, W═C, J=CH₂, Z=CD₃ as a hydrochloride salt as a brownish powder.

### EXAMPLE 30

### Precipitation of the labeled CPT-11 (16) as a free base.

The crude material containing the compound labeled CPT-11 (16) as a hydrochloride salt, prepared as described in EXAMPLE 29, was dissolved in water (9.0 ml) and the value of the pH was adjusted to 7.0 by adding di-Potassium hydrogen phosphate. The precipitated free base of the labeled CPT-11 (16) was isolated by filtration and washed with water (20 ml).

### EXAMPLE 31

### Precipitation of the Labeled CPT-11 (16) as a Hydrochloride Salt.

To a solution of the crude free base of the compound of the labeled CPT-11 prepared as described in EXAMPLE 30, in waters. (25.0 ml), 1N hydrochloric acid (1.31 ml, about 1.3 equivalents) was added. The acidic solution was filtered and the filtrate was evaporated under reduced pressure at 40° C. to a smaller volume (about 2.8 ml). The labeled CPT-11 (16) as hydrochloride salt was isolated from the aqueous solution by freeze-drying. The labeled CPT-11 (16) was obtained as a white solid (256 mg). The purity greater than 98.8% was assessed by HPLC (on C18 reverse phase column along with eluant as mixture of water-acetonitrile-trifluoroacetic acid in a constant ratio of 71:29:0.2 by volume at a flow rate of 1 0.7 ml/minute), the retention time (Rt=7.40 minutes) was the same as the retention time of an authentic non-labeled sample. The mass spectrum of the title compound was recorded using the electrospray ionization technique (ESI) with positive ion detection. The ESI mass spectrum showed the protonated molecular ions at m/z 600 amu. The NMR spectrum recorded in DMSO-d5 at 500 MHz showed the following signals expressed as chemical shifts (ppm): 9.94, b; 8.20, d; 8.00, d; 7.69, dd; 7.33, s; 6.52, b; 5.44, s; 5.35, s; 4.42, m; 4.21, m; 3.44, m; 3.18, s; 2.95-3.15, m; 2.17, m; 1.67-1.95, m; 0.89, t.

### EXAMPLE 32

### Compound of Formula (I″) Where Q=CH₂, B₁═N, Y═C, X═CH, W═C, J-=CH₂, Z=CD₃, Labeled CPT-11 (18).

Starting from the labeled SN-38 of formula (1′) prepared as described in EXAMPLE 4 and purified as described in EXAMPLE 5, and the compound of formula (VII′) where Q=CH₂, B₁═N, and following the procedure described in EXAMPLES 29 to 30, titled compound can be obtained as a free base. The corresponding hydrochloride salt can be obtained by following the procedure described in EXAMPLE 31.

### EXAMPLE 33

### Compound of Formula (I″) Where Q=CH₂, B₁═NH, Y═C, X═CH, W═C, J=CH₂, Z=CD₃, Labeled CPT-11 (17).

Starting from the labeled SN-38 of formula (1′) prepared as described in EXAMPLE 4 and purified as described in EXAMPLE 5, and the labeled compound of formula (VII′) where Q=CH₂, Bₗ═¹⁵NH, prepared as described in EXAMPLE 25 and following the procedure described in EXAMPLES 29 to 30, titled compound can be obtained as a free base. The corresponding hydrochloride salt can be obtained by following the procedure described in EXAMPLE 31.

### EXAMPLE 34

### Compound of Formula (I″) Where Q=CD₂, B₁═N, Y═¹³C, X═¹³CH, W═C, J=CH₂, Z=CD₃, Labeled CPT-11 (19).

Starting from the labeled SN-38 of formula (2′) prepared as described in EXAMPLE 6, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 35

### Compound of Formula (I″) Where Q=CH₂, B₁═N, Y═¹³C, X═³CH, W═C, J=CH₂, Z=CD₃, Labeled CPT-11 (21).

Starting from the labeled SN-38 of formula (2) prepared as described in EXAMPLE 6, and the compound of formula (VII′) where Q=CH₂, B₁═N, and following the procedure described in EXAMPLES 29 to 30, the tilted compound can be obtained as a free base. The corresponding hydrochloride salt can be obtained by following the procedure described in EXAMPLE 31.

### EXAMPLE 36

### Compound of Formula (I″) Where Q=CH₂₁ B₁═¹⁵N, Y═¹³C, X═¹³CH, W═C, J=CH₂, Z=CD₃, Labeled CPT-11 (20).

Starting from the labeled SN-38 of formula (2) prepared for example as described in EXAMPLE 6, and the labeled compound of formula (VII′) where Q=CH2, B₁═32 ¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 37

### Compound of Formula (I″) Where Q=CD₂, B₁═N, Y═C, X═CH, W═C, J=CD₂, Z=CD₃, Labeled CPT-11 (22).

Starting from the labeled SN-38 of formula (3) prepared as described in EXAMPLE 7, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 38

### Compound of Formula (I″) Where Q=CH₂, B₁═N, Y═C, X═CH, W═C, J=CD₂, Z=CD₃, Labeled CPT-11 (24).

Starting from the labeled SN-38 of formula (3) prepared as described in EXAMPLE 7, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 39

### Compound of Formula (I″) Where Q=CH₂, B₁═¹⁵N, Y═C, X═CH, W═C, J=CD₂, Z=CD₃, Labeled CPT-11 (23).

Starting from the labeled SN-38 of formula (3) prepared as described in EXAMPLE 7, and the labeled compound of formula (VII′) where Q=CH₂, B₁═¹⁵N, prepared as described in EXAMPLE 25 and following for example the procedure described in EXAMPLES 29 to 30, the titled compound can be obtained as a free base. The corresponding hydrochloride salt can be obtained by following the procedure described in EXAMPLE 31.

### EXAMPLE 40

### Compound of Formula (I″) Where Q=CD₂, B₁═N, Q=CH₂, B₁═NH, Y═¹³C, X═¹³CH, W═C, J=CD₂, Z=CD₃, Labeled CPT-11 (25).

Starting from the labeled SN-38 of formula (4) prepared as described in EXAMPLE 8, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 41

### Compound of Formula (I″) Where Q=CH₂, B₁═N, Y═¹³C, X═¹³CH, W═C, J=CD₂, Z=CD₃, Labeled CPT-11 (27).

Starting from the labeled SN-38 of formula (4) prepared as described in EXAMPLE 8, and the compound of formula (VII′) where Q=CH₂, B₁═N, and following the procedure described in4 EXAMPLES 29 to 30, the titled compound can be obtained as a, free base. The corresponding hydrochloride salt can be obtained by following the procedure described in EXAMPLE 31.

### EXAMPLE 42

### Compound of Formula (I″) Where Q=CH₂, B₁═¹⁵N, Y═¹³C, X═¹³CH, W═C, J=CD₂, Z=CD₃, Labeled CPT-11 (26).

Starting from the labeled SN-38 of formula (4) prepared as described in EXAMPLE 8, and the labeled compound of formula (VII′) where Q=CH₂, B₁═¹⁵N, prepared as described in EXAMPLE 25 and following the procedure described in EXAMPLES 29 to 30, tilted compound can be obtained as a free base. The corresponding hydrochloride salt can be obtained by following the procedure described in EXAMPLE 31.

### EXAMPLE 43

### Compound of Formula (I″) Where Q=CD₂, B₁═N, Q2=CH₂, B2═NH, Y═¹³C, X═¹³CH, W═¹³C, J=¹³CH₂, Z=¹³CH₃, Labeled CPT-11 (28).

Starting from the labeled SN-38 of formula (5) prepared as described in EXAMPLE 9, and the labeled compound of formula (VII′) where Q=CD₂, B₁═N prepared as described in EXAMPLE 24 and following the procedure described in EXAMPLES 29 to 30, tit ed compound can be obtained as a free base. The corresponding hydrochloride salt can be obtained by following the procedure described in EXAMPLE 31.

### EXAMPLE 44

### Compound of Formula (I″) Where Q=CH₂, B₁═N, Y═¹³C, X═¹³CH, W═¹³C, J=¹³CH₂, Z=¹³CH₃, Labeled CPT-11 (30).

Starting from the labeled SN-38 of formula (5) prepared as described in EXAMPLE 9, and the compound of formula (VII′) where Q=CH₂, B₁═N and following the procedure described in EXAMPLES 29 to 30, the tilted compound can be obtained as a free base. The corresponding hydrochloride salt can be obtained by following the procedure described in EXAMPLE 31.

### EXAMPLE 45

### Compound of Formula (I″) Where Q=CH₂, B₁═¹⁵N, Y═¹³C, X═¹³CH, W═¹³C, J=¹³CH₂, Z=¹³CH₃, Labeled CPT-11 (29).

Starting from the labeled SN-38 of formula (5) prepared as described in EXAMPLE 9, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 46

### Compound of Formula (I″) Where Q=CD₂, B₁═N, Y═C, X═CH, W═¹³C, J=¹³CH₂, Z=¹³CH₃, Labeled CPT-11 (31).

Starting from the labeled SN-38 of formula (6) prepared as described in EXAMPLE 10, and the labeled compound of formula (VII′) where Q=CD₂, B₁═N, prepared as described in EXAMPLE 24 and following the procedure described in EXAMPLES 29 to 30, the titled can be obtained as a free base. The corresponding hydrochloride salt can be obtained by following the procedure described in EXAMPLE 31.

### EXAMPLE 47

### Compound of Formula (I″) Where Q=CH₂, B₁═N, Y═C, X═CH, W═¹³C, J=¹³CH₂, Z=¹³CH₃, Labeled CPT-11 (33).

Starting from the labeled SN-38 of formula (6) prepared as described in EXAMPLE 10, and the compound of formula (VII′) where Q=CH₂, B₁═N and following the procedure described in EXAMPLES 29 to 30, the titled can be obtained as a free base. The corresponding hydrochloride salt can be obtained by following for example the procedure described in EXAMPLE 31.

### EXAMPLE 48

### Compound of Formula (I″) Where Q=CH₂, B₁═¹⁵N, Y═C, X═¹H, W═¹³C, J=¹³CH₂, Z=¹³CH₃, Labeled CPT-11 (32).

Starting from the labeled SN-38 of formula (6) prepared as described in EXAMPLE 10, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 49

### Compound of Formula (I″) Where Q=CD₂, B₁═N, Y═¹³C, X═¹³CH, W═C, J=CH₂, Z=CH₃, Labeled CPT-11 (34).

Starting from the labeled SN-38 of formula (7) prepared as described in EXAMPLE 11, and the labeled compound of formula (VII′) where Q=CD₂, B₁═2 N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 50

### Compound of Formula (I″) Where Q=CH₂, B₁═N, Y═¹³C, X═¹³CH, W═C, J=CH₂, Z=CH₃, Labeled CPT-11 (36).

Starting from the labeled SN-38 of formula (7) prepared, as described in EXAMPLE 11, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 51

### Compound of Formula (I″) Where Q=CH₂, B₁═¹⁵N, Y═¹³C, X═¹³CH, W═C, J=CH₂, Z=CH₃, Labeled CPT-11 (35).

Starting from the labeled SN-38 of formula (7) prepared as described in EXAMPLE 11, and the labeled compound of formula (VII′) where Q=CH₂, B₁═¹⁵N, Q2=CH₂, B2═NH, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 52

### Compound of Formula (I″) Where Q=CD₂, B₁═N, Y═¹³C, X═¹³CH, W═C, J=¹³CH₂, Z=CH₃, Labeled CPT-11 (37).

Starting from the labeled SN-38 of formula (8) prepared as described in EXAMPLE 12, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 53

### Compound of Formula (I″) Where Q=CH₂, B₁═N, Y═¹³C, X═¹³CH, W═C, J=¹³CH₂, Z=CH₃, Labeled CPT-11 (39).

Starting from the labeled SN-38 of formula (8) prepared as described in EXAMPLE 12, and the compound of formula (VII′) where Q=CH₂, B₁═N and following the procedure described in EXAMPLES 29 to 30, the titled compound can be obtained as a free base. The corresponding hydrochloride salt can be obtained by following the procedure described in EXAMPLE 31.

### EXAMPLE 54

### Compound of Formula (I″) Where Q=CH₂, B₁═¹⁵N, Y═¹³C, X═¹³CH, W═C, J=¹³CH₂, Z=CH₃, Labeled CPT-11 (38).

Starting from the labeled SN-38 of formula (8) prepared as described in EXAMPLE 12, and the labeled compound of formula (VII′) where Q=CH₂, B₁═¹⁵N, prepared as described in EXAMPLE 25 and following the procedure described in EXAMPLES 29 to 30, the titled compound can be obtained: as a free base. The corresponding hydrochloride salt can be obtained by following the procedure described in EXAMPLE 31.

### EXAMPLE 55

### Compound of formula (I″) Where Q=CD₂, B₁═N, Y═¹³C, X═¹³CH, W═C, J=CH₂, Z=¹³CH₃, Labeled CPT-11 (40).

Starting from the labeled SN-38 of formula (9) prepared as described in EXAMPLE 13, and the labeled compound of formula (VII′) where Q=CD₂, B₁=N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 56

### Compound of formula (I″) Where Q=CH₂, B₁═N, Y═¹³C, X═¹³CH, W═C, J=CH₂, Z=¹³CH₃, Labeled CPT-11 (42).

Starting from the labeled SN-38 of formula (9) prepared as described in EXAMPLE 13, and the compound of formula (VII′) where Q=CH₂, B₁═N, Q2=CH₂, B₂═NH, and following the procedure described in EXAMPLES 29 to 30, the titled compound can be obtained as a free base. The corresponding hydrochloride salt can be obtained by following the procedure described in EXAMPLE 31.

### EXAMPLE 57

### Compound of Formula (I″) Where Q=CH₂, B₁═¹⁵N, Y═¹³C, X═¹³ CH, W═C, J=CH₂, Z=¹³CH₃, Labeled CPT-11 (41).

Starting from the labeled SN-38 of formula (9) prepared as described in EXAMPLE 13, and the labeled compound of formula (VII′) where Q=CH₂, B=═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 58

### Compound of Formula (I″) Where Q-CD₂, B₁═N, Y═¹³C, X═¹³CH, W═¹³C, J=CH₂, Z=CH₃, Labeled CPT-11 (43).

Starting from the labeled SN-38 of formula (10) prepared as described in EXAMPLE 14, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 59

### Compound of formula (I″) Where Q=CH₂, B₁═N, Y═¹³C, X═¹³CH, W═¹³C, J=CH₂, Z=CH₃, Labeled CPT-11 (45).

Starting from the labeled SN-38 of formula (10) prepared as described in EXAMPLE 14, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 60

### Compound of Formula (I″) Where Q=CH₂, B₁═¹⁵N, Y═¹³C, X═¹³CH, W═¹³C, J=CH₂, Z=CH₃, Labeled CPT-11 (44).

Starting from the labeled SN-38 of formula (10) prepared as described in EXAMPLE 14, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE-61

### Labeled CPT-11 (46) Where Q=CD₂, B1═N, Y═C, X═CH, W═¹³C, J=CH₂, Z=CH₃

Starting from the labeled SN-38 of formula (11) where Z=CH₃, J=CH₂, W═¹³C, X═CH, Y═C, prepared for example as described in EXAMPLE 15, and the labeled compound of formula (VII′) where Q=CD₂, B₁═N, prepared for example as described in EXAMPLE 24 and following for example the procedure described in EXAMPLES 29 to 30, the labeled CPT-11 of the formula (46) where Q=CD₂, B₁═N, Y═C, X═CH, W═¹³C, J=CH₂, Z=CH₃ can be obtained as a free base. The corresponding hydrochloride salt can be obtained by following for example the procedure described in EXAMPLE 31.

### EXAMPLE 62

### Compound of Formula (I″) Where Q=CH₂, B₁═N, Y═C, X═CH, W═¹³C, J=CH₂, Z=CH₃, Labeled CPT-11 (48).

Starting from the labeled SN-38 of formula (11) prepared as described in EXAMPLE 15, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 63

### Compound of Formula (I″) Where Q=CH₂, B₁═¹⁵N, Y═C, X═CH, W═¹³C, J=CH₂, Z=CH₃, Labeled CPT-11 (47).

Starting from the labeled SN-38 of formula (11) prepared as described in EXAMPLE 15, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 64

### Compound of Formula (I″) Where Q=CD₂, B₁═N, Y═C, X═CH, W═C, J=¹³CH₂, Z=CH₃, Labeled CPT-11 (49).

Starting from the labeled SN-38 of formula (12) prepared as described in EXAMPLE 16, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 65

### Compound of Formula (I″) Where Q=CH₂, B₁═N, Y═C, X═CH, W═C, J=¹³CH₂, Z=CH₃, Labeled CPT-11 (51).

Starting from the labeled SN-38 of formula (12) prepared as described in EXAMPLE 16, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 66

### Compound of Formula (I″) Where Q=CH₂, B₁═¹⁵N, Y═C, X═CH, W═C, J=¹³CH₂, Z=CH₃, Labeled CPT-11 (50).

Starting from the labeled SN-38 of formula (12) prepared as described in EXAMPLE 16, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 67

### Compound of Formula (I″) Where Q=CD₂, B1═N, Y═C, X═CH, W═C, J=CH₂, Z=¹³CH₃ Labeled CPT-11 (52).

Starting from the labeled SN-38 of formula (13) prepared as described in EXAMPLE 17, and the labeled compound of formula (VII′) where Q=CD₂, B₁═N, prepared as described in EXAMPLE 24 and following the procedure described in EXAMPLES 29 to 30, the titled compound can be obtained as a free base. The corresponding hydrochloride salt can be obtained by following the procedure described in EXAMPLE 31.

### EXAMPLE 68

### Compound of Formula (I″) Where Q=CH₂, B₁═N, Y═C, X═CH, W═C, J=CH₂, Z=¹³CH₃, Labeled CPT-11 (54).

Starting from the labeled SN-38 of formula (13) prepared as described in EXAMPLE 17, and the compound of formula (VII′) where Q=CH₂, B₁═N, and following the procedure described in EXAMPLES 29 to 30, the titled compound can be obtained as a free base. The corresponding hydrochloride salt can be obtained by following the procedure described in EXAMPLE 31.

### EXAMPLE 69

### Compound of Formula (I″) Where Q=CH₂, B₁═¹⁵N, Y═C, X═CH, W═C, J=CH₂, Z=¹³CH₃, Labeled CPT-11 (53).

Starting from the labeled SN-38 of formula (13) prepared as described in EXAMPLE 17, and the labeled compound of formula (VII′) where Q=CH₂, B₁═¹⁵N, prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

### EXAMPLE 70

### Compound of Formula (I″) Where Q=CH₂, B₁═¹⁵N, Y═C, X═CH, W═C, J=CH₂, Z=CH3, Labeled CPT-11 (15).

Starting from the compound SN-38 and the labeled compound of formula (VII′) prepared as described in EXAMPLE 25 and following the procedure described in EXAMPLES 29 to 30, the titled compound can be obtained as a free base. The corresponding hydrochloride salt can be obtained by following the procedure described in EXAMPLE 31.

1. A stable labeled camptothecin analogs of formula (I) 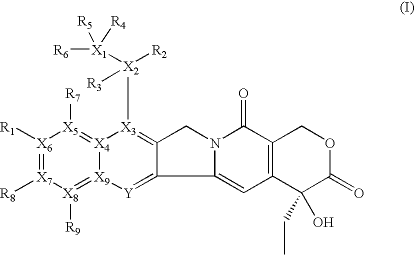
wherein
each of R₂, R₃, R₄, R₅, R₆, R₇, R₈ and R₉ independently represents ²H or H;
each of X₁, X₂, X₃, X₄, X₅, X₆, X₇, X₈ and X₉ independently represents ¹³C or C;
Y is ¹⁵N or N; and
R₁ is a hydroxyl group or a group of formula (i) 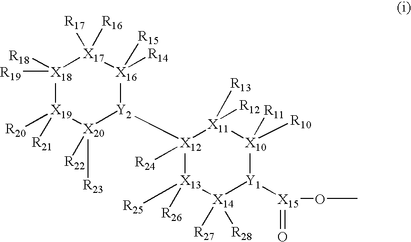
wherein
each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,
each of X₁₀, X₁₁, X₁₂, X₁₃, X₁₄, X₁₅, X₁₆, X₁₇, X₁₈, X₁₉ and X₂₀ independently represents ¹³C or C,
each of Y₁ and Y₂ independently represents ¹⁵N or N; with the proviso that at least one of R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, Y, Y₁ and Y₂ is isotopically labeled; or a pharmaceutically acceptable salt thereof.

2. A compound of formula (I) as claimed in claim 1, wherein R₁ is a hydroxyl group.

3. A compound of formula (I) as claimed in claim 1, wherein R₁ is a group of formula (i) as defined in claim 1.

4. A compound of formula (I) as claimed in claim 1, wherein R₂, R₃, R₄, R₅, R₆, R₇, R₈ and R₉ are all H, X₁, X₂, X₃, X₄, X₅, X₆, X₇, X₈ and X₉ are all C, Y is N and R₁ is a group (i) as defined in claim 1.

5. A compound of formula (I) as claimed in claim 1, wherein each of R₂, R₃, R₄, R₅, R₆, R₇, R₈ and R₉ independently represents ²H of H, each of X₁, X₂, X₃, X_{4,} X₅, X₆, X₇, X₈ and X₉ independently represents ¹³C or C, Y is ¹⁵N or N, R₁ is a hydroxyl group or a group of formula (i) wherein R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ are all H, X₁₀, X₁₁, X₁₂, X₁₃, X₁₄, X₁₅, X₁₆, X₁₇, X₁₈, X₁₉ and X₂₀ are all C and Y₁ and Y₂ are N.

6. A compound of formula (I′) 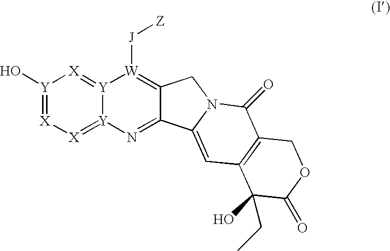
as defined in TABLE 1.

7. A compound of formula (I″), optionally in the form of a pharmaceutical acceptable salt, 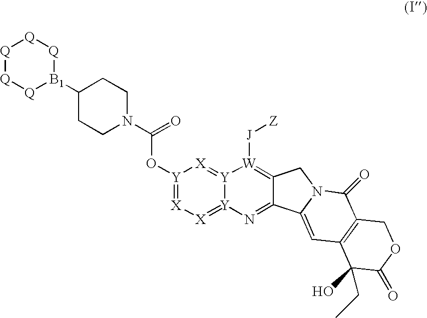
as defined in TABLE 2.

8. A process for the preparation of a stable labeled camptothecin analog of formula (I) as defined in claim 1, wherein
R₁ is a hydroxyl group,
each of R₂, R₃, R₄, R₅, R₆, R₇, R₈ and R₉ independently represents ²H or H,
each of X₃, X₂, X₃, X₄, X₅, X₆, X₇, X₈ and X₉ independently represents ¹³C or C, and
Y is ¹⁵N or N,
with the proviso that at least one of R₂, R₃, R₄, R₅, R₆, R₇, R₈, R₉, X₁, X₂, X₃, X₄, X₅, X₆, X₇, X₈, X₉ and Y is isotopically labeled,
which comprises:
(a) reacting a compound of formula (II) 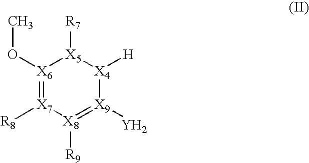
wherein
each of R₇, R₈ and R₉ independently represents ²H or H,
each of X₄, X₅, X₆, X₇, X₈ and X₉ independently represents ¹³C or C, and
Y is ¹⁵N or N,
with a compound of formula (III) 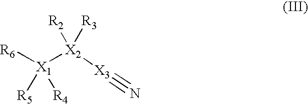
wherein
each of R₂, R₃, R₄, R₅ and R₆ independently represents ²H or H, and
each of X₁, X₂ and X₃ independently represents ¹³C or C, to obtain the compound of formula (IV) 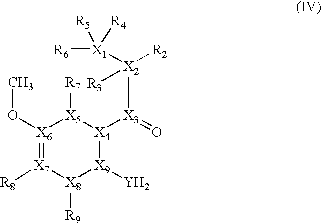
wherein
each of R₂, R₃, R₄, R₅, R₆, R₇, R₈, R₉, X₁, X₂, X₃, X₄, X₅, X₆, X₇, X₈, X₉ and Y, are as above described,
so that at least one of R₂, R₃, R₄, R₅, R₆, R₇, R₈, R₉, X₁, X₂, X₃, X₄, X₅, X₆, X₇, X₈, X₉ and Y is isotopically labeled;
(b) cleaving a compound of formula (IV) to obtain a compound of formula (V) 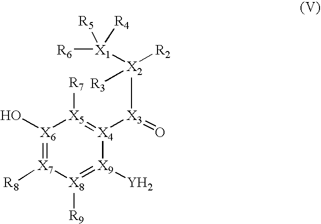
wherein
R₂, R₃, R₄, R₅, R₆, R₇, R₈, R₉, X₁, X₂, X₃, X₄, X₅, X₆, X₇, X₈, X₉ and Y are as above described for the compound (IV); and
(c) reacting a compound of formula (V) with the compound of formula (VI) 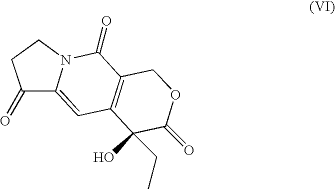
to obtain the desired compound of formula (I).

9. A process for preparing a compound of formula (I) as defined in claim 1, wherein
each of R₂, R₃, R₄, R₅, R₆, R₇, R₈ and R₉ independently represents ²H or H,
each of X₁, X₂, X₃, X₄, X₅, X₆, X₇, X₈ and X₉ independently represents ¹³C or C,
Y is ¹⁵N or N, and
R₁ is a group of formula (i) 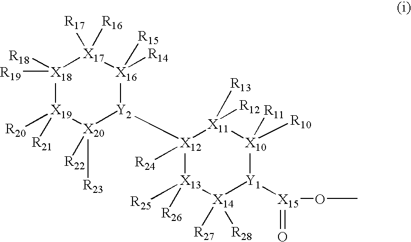
wherein
each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,
each of X₁₀, X₁₁, X₁₂, X₁₃, X₁₄, X₁₅, X₁₆, X₁₇, X₁₈, X₁₉ and H₂₀ independently represents ¹³C or C, and
each of Y₁ and Y₂ independently represents ¹⁵N or N,
with the proviso that at least one of R₂, R₃, R₄, R₅, R₆, R₇, R₈, R₉, X₁, X₂, X₃, X₄, X₅, X₆, X₇, X₈, X₉ and Y is isotopically labeled, and that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀,
Y₁ and Y₂ is isotopically labeled,
which comprises:
(d) reacting a compound of formula (I) as obtained in step (c) above with a compound of formula (VII) 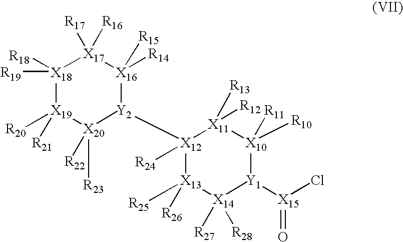
wherein
each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄ R₂₅, R₂₆, R₂₇ and R₂₈ independentently represents ²H or H.
each of X₁₀, X₁₁, X₁₂, X₁₃, X₁₄, X₁₅, X₁₆, X₁₇, X₁₈ and X₂₀ independently represents ¹³C or C, and
each of Y₁ and Y₂ independently represents ¹⁵N or N,
with the proviso that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀, Y₁ and Y₂ is isotopically labeled, to obtain the desired compound of formula (I).

10. A process for preparing a compound of formula (I) as defined in claim 1, wherein R₂, R₃, R₄, R₅, R₆, R₇, R₈ and R₉ are all H; X₁, X₂, X₃, X₄, X₅, X₆, X₇, X₈, and X₉ are all C, Y is N and R₁ is a group of formula (i) 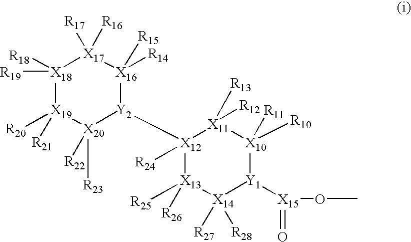
wherein
each of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇ and R₂₈ independently represents ²H or H,
each of X₁₀, X₁₁, X₁₂, X₁₃, X₁₄, X₁₅, X₁₆, X₁₇, X₁₈, X₁₉ and X₂₀ independently represents ¹³C or C, and
each of Y₁ and Y₂ independently represents ¹⁵N or N,
with the proviso that at least one of R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, X₁₀, X₁₁, X₁₂, X₁₃, X₁₄, X₁₅, X₁₆, X₁₇, X₁₈, X₁₉, X₂₀, Y₁ and Y₂ is isotopically labeled,
which comprises:
(e) reacting the compound of formula 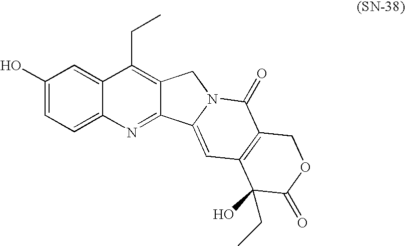
with a compound of formula (VII) as above described to obtain the desired compound of formula (I), and optionally converting it into a pharmaceutically acceptable salt thereof.

11. A process for preparing a compound of formula (I) as defined in claim 1, wherein
each of R₂, R₃, R₄, R₅, R₆, R₇, R₈, R₉, X₁, X₂, X₃, X₄, X₅, X₆, X₇, X₈, X₉ and Y, are as above described, with the proviso that at least one of R₂, R₃, R₄, R₅, R₆, R₇, R₈, R₉, X₁, X₂, X₃, X₄, X₅, X₆, X₇, X₈, X₉ and Y is isotopically labeled, and
R₁ is a group of formula (i) wherein R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ are all H and X₁₀, X₁₁, X₁₂, X₁₃, X₁₄, X₁₅, X₁₆, X₁₇, X₁₈, X₁₉ and X₂₀ are all C, Y₁ and Y₂ are N,
which comprises:
(f) reacting a compound of formula (I) as obtained in step (c) above with the compound of formula 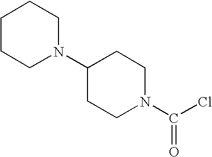
to obtain the desired compound of formula (I), and optionally converting it into a pharmaceutically acceptable salt thereof.

12. Use of a stable labeled camptothecin analog of formula (I) as claimed in claim 1, for ADME studies.

13. Use of a stable labeled camptothecin analog of formula (I) as claimed in claim 1, as an internal standard in an analytical method for the quantitative detection of the corresponding unlabeled camptothecin analog in a biological sample.

14. Use of a stable labeled camptothecin analog of formula (I′) as claimed in claim 6 and formula (I″) as claimed in claim 7 or a pharmaceutically acceptable salt thereof as an internal standard in an analytical method for the quantitative detection of the corresponding unlabeled camptothecin analog in a biological sample.
